(19) United States
(12) Patent Application Publication
(10) Pub. No.: US 2025/0136618 A1
(43) Pub. Date: May 1, 2025

(54) ANTIBIOTIC PYRAZINOTHIAZINE DERIVATIVES AND PROCESS OF PREPARATION THEREOF

(71) Applicant: Bugworks Research India PVT Ltd, Bengaluru (IN)

(72) Inventors: Shahul Hameed Peer Mohamed, Bengaluru (IN); Ranga Rao Kajipalya Ranganatha Rao, Bengaluru (IN); Nagakumar Bharatham, Bengaluru (IN); Nainesh Katagihalli Math, Bengaluru (IN); Sreevalli Sharma, Bengaluru (IN); Radha Nandishaiah, Bengaluru (IN); Vasanthi Ramachandran, Bengaluru (IN)

(73) Assignee: Bugworks Research India PVT Ltd, Bengaluru (IN)

(21) Appl. No.: 18/682,941

(22) PCT Filed: Aug. 12, 2022
(86) PCT No.: PCT/IN2022/050732

§ 371 Date: Feb. 12, 2024

(30) Foreign Application Priority Data
Aug. 13, 2021 (IN) 202141036833

(51) Int. Cl. C07D 519/00; A61K 31/542; A61P 31/04

(52) U.S. Cl. CPC C07D 519/00; A61K 31/542; A61P 31/04

(57) ABSTRACT

The present disclosure provides a compound selected from Formula Ia or Formula Ib, its stereoisomers, pharmaceutically acceptable salts, complexes, hydrates, solvates, tautomers, polymorphs, or pharmaceutically active derivatives thereof. The compounds of the present disclosure are antibiotic compounds which are effective in killing and inhibiting growth of microorganisms. The present disclosure also provides a process for preparation of the compounds and methods thereof.

### FIELD OF INVENTION

The present disclosure relates to the field of medicinal chemistry and more particularly to antimicrobial compounds, in particular compounds of Formula Ia and Tb, its stereoisomers, pharmaceutically acceptable salts, complexes, hydrates, solvates, tautomers, polymorphs, or pharmaceutically active derivatives thereof and pharmaceutical compositions containing them as the active ingredient. The present disclosure further relates to a process of preparation of the aforementioned compounds.

The compounds of the present disclosure are useful as medicaments for the treatment, prevention or suppression of diseases, and conditions mediated by microbes.

### BACKGROUND

With the rapidly increasing use of antibiotics in various medical treatments, the risk of development of antibiotic resistant infections has also increased. The emergence of new resistance mechanisms in many commonly occurring disease causing microorganisms has limited our ability to treat common infections such as tuberculosis, pneumonia, food poisoning, and the like. The unreasonable clinical misuse and cutbacks in drug research further aid in making the situation worse. Many of the presently available antibacterial drugs have been found to become ineffective against even the most commonly occurring bacteria such as Acinetobacter baumannii, Staphylococcus aureus and Escherichia coli.

Thus, there is a dire need in the present state of the art to develop new compounds with improved antibacterial activity, higher selectivity against target microorganisms and reduced tendency for developing antibacterial resistance.

### SUMMARY OF THE INVENTION

The present disclosure relates to a compound selected from Formula Ia or Formula Tb, its stereoisomers, pharmaceutically acceptable salts, complexes, hydrates, solvates, tautomers, polymorphs, or pharmaceutically active derivatives thereof,

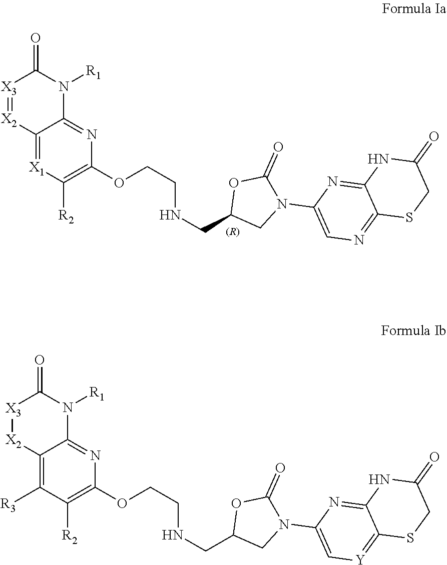

wherein
R₁ is selected from C₁₋₆ alkyl, C₂₋₆ alkenyl, C₃₋₆ cycloalkyl, CD₃, C₁₋₆ alkoxy, C₁₋₆ haloalkyl, or C₁₋₆ haloalkoxy;
R₂ is selected from hydrogen, C₁₋₆ alkyl, halogen, hydroxy, or amino;
R₃ is selected from hydrogen, halogen, hydroxyl, amino, cyano, C₁₋₆ alkoxy, C₁₋₆ haloalkyl, C₁₋₆ haloalkoxy, C₁₋₆ alkyl, NH—R₄, or —CH₂CH₂OH;
R₄ is selected from C₁₋₆ alkyl, C₃₋₆ cycloalkyl, C₁₋₆ alkoxy, C₁₋₆ haloalkyl, C₁₋₆ haloalkoxy —CH₂CH₂OH, or —CH₂CH₂NH₂;
X₁ is N or CR₃;
X₂ is CR₅, O, N, or NR₆ when X₃ is CH or CH₂;
R₅ is selected from hydrogen, cyano, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl;
R₆ is selected from hydrogen, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl;
X₃ is N or NR₇ when X₂ is CH₂ or CR₅;
R₇ is selected from hydrogen, or C₁₋₆ alkyl;
Y is N or CR₈; and
R is selected from hydrogen, halogen, cyano, C₁₋₆ alkyl, C₁₋₆ alkoxy, or C₁₋₆ haloalkoxy.

The present disclosure also relates to a process of preparation of compounds of Formula Ia, its pharmaceutically acceptable salts, complexes, hydrates, solvates, tautomers, polymorphs, or pharmaceutically active derivative thereof as disclosed herein, said process comprising reacting Formula (X) with Formula (VI) in presence of at least one reducing agent to obtain the compounds of Formula Ia.

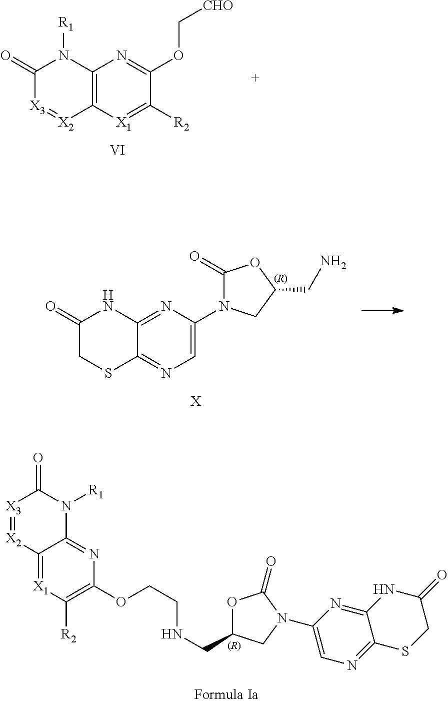

The present disclosure also relates to a process of preparation of compounds of Formula Ib, its stereoisomers, pharmaceutically acceptable salts, complexes, hydrates, solvates, tautomers, polymorphs, or pharmaceutically active derivative thereof as disclosed herein, said process comprising reacting Formula (XI) with Formula (VII) in presence of at least one reducing agent to obtain the compounds of Formula Ib

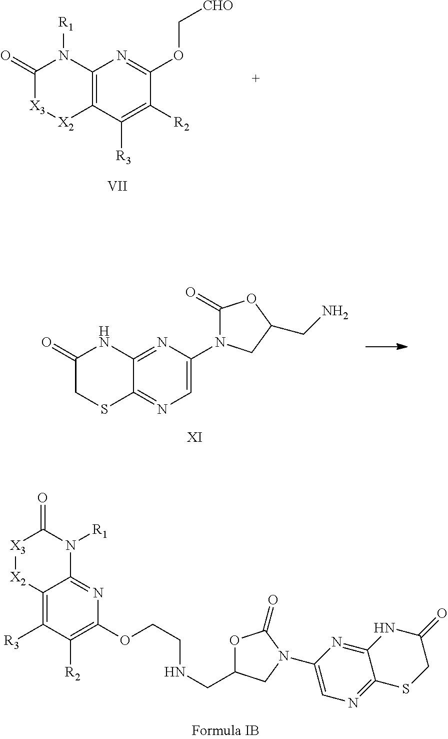

The present disclosure further relates to a pharmaceutical composition comprising a compound selected from Formula Ia or Formula Tb, its stereoisomers, pharmaceutically acceptable salts, complexes, hydrates, solvates, tautomers, polymorphs, or pharmaceutically active derivative thereof as disclosed herein, together with a pharmaceutically acceptable carrier, optionally in combination with at least one antibiotic.

The present disclosure further relates to a pharmaceutical composition comprising a compound selected from Formula Ia or Formula Tb, its stereoisomers, pharmaceutically acceptable salts, complexes, hydrates, solvates, tautomers, polymorphs, or pharmaceutically active derivative thereof as disclosed herein, wherein the compound selected from Formula Ta or Formula Tb has enantiomeric excess in the range of 95% to 99.9%.

The present disclosure further relates to use of compound selected from Formula Ta or Formula Tb, its stereoisomers, pharmaceutically acceptable salts, complexes, hydrates, solvates, tautomers, polymorphs, or pharmaceutically active derivative thereof as disclosed herein, in killing or inhibiting the growth of a microorganism selected from the group consisting of bacteria, virus, fungi, and protozoa.

The present disclosure further relates to a method for treatment of bacterial infection in a subject comprising: administering to the subject an effective amount of the compound selected from Formula Ta or Formula Tb, its stereoisomers, pharmaceutically acceptable salts, complexes, hydrates, solvates, tautomers, polymorphs, or pharmaceutically active derivative thereof as disclosed herein, wherein the said bacterial infection is caused by a gram-positive or a gram-negative pathogen.

These and other features, aspects, and advantages of the present subject matter will become better understood with reference to the following description. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION

Those skilled in the art will be aware that the present disclosure is subject to variations and modifications other than those specifically described. It is to be understood that the present disclosure includes all such variations and modifications.

The disclosure also includes all such steps, features, compositions and compounds referred to or indicated in this specification, individually or collectively, and any and all combinations of any or more of such steps or features.

### Definitions

For convenience, before further description of the present disclosure, certain terms employed in the specification, and examples are collected here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. Throughout this specification, unless the context requires otherwise the word “comprise”, and variations, such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

In the structural formulae given herein and throughout the present disclosure, the following terms have been indicated meaning, unless specifically stated otherwise.

The term “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge and expertise of the attending physician.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methane sulphonic, ethane sulphonic, benzene sulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

The compounds discussed herein in many instances may have been named and/or checked with ACD/Name by ACD/Labs® and/or Chemdraw by CambridgeSoft®.

The term “polymorphs” refers to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice.

Salts and solvates having non-pharmaceutically acceptable counter-ions or associated solvents are within the scope of the present disclosure, for example, for use as intermediates in the preparation of other compound of Formula Ia or Formula Ib, and their pharmaceutically acceptable salts. Thus, one embodiment of the disclosure embraces compound of Formula Ta or Formula Ib, and salts thereof. Compounds according to Formula Ta or Formula Ib contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenyl acetate, propionate, butyrate, iso-butyrate, valerate, maleate, hydroxymaleate, acrylate, fumarate, malate, tartrate, citrate, salicylate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), aminobenzenesulfonate, p-toluenesulfonate (tosylate), and naphthalene-2-sulfonate.

The term “solvate”, as used herein, refers to a crystal form of a substance which contains solvent.

The term “complexes” as used herein, can be interchangeably used as “coordination complex,” or “metal coordination complex,” and the like. It refers to a complex of an organic compound with a metal that can be empirically differentiated from a simple metal salt of the organic compound based on physiochemical and/or spectroscopic properties, with a coordination complex typically having enhanced covalency as compared to a salt. Without limitation “complexes” as used herein also involves a combination of coordinate covalent bonds and/or ionic bonds. As used herein, the term “complexes” also includes molecules that lack an ionic component (e.g., such as a neutral coordination complex prior to deprotonation, where pKa of the coordination complex falls within a physiologically acceptable range).

The term “hydrate” refers to a solvate wherein the solvent is water.

The compounds provided herein, includes the corresponding enantiomers and stereoisomers, that is, the pure form of the stereoisomers, in terms of geometrical isomer, enantiomer, or diastereomer, and the mixture of enantiomeric and stereoisomeric form of said compounds. Further, the mixture of enantiomeric and stereoisomeric forms can be resolved into their pure component by the methods known in the art, such as chiral-phase gas chromatography, chiral-phase high performance liquid chromatography, crystallization, using chiral derivatizing agents, etc. Also, the pure enantiomers and stereoisomers can be obtained from intermediates or metabolites and reagents that are in the form of pure enantiomers and stereoisomers by known asymmetric synthetic methods.

The term “enantiomers” refers to the stereoisomers of the compound selected from Formula Ia or Formula Ib that are non-superimposable mirror images of each other. Enantiomeric excess refers to the absolute difference between each enantiomer. Enantiomeric excess refers to chiral compounds having more of one enantiomer than the other. The compound of Formula Ia or Formula Ib has enantiomeric excess in the range of 95 to 99.9% which refers to one of the stereoisomer is higher and also refers that the compounds are enantiomerically pure. In case of Formula Ib, the enantiomeric excess can be with respect to (R)-isomer or (S)-isomer independently.

The terms “bacterium”, “bacteria”, and “pathogens” have been used interchangeably throughout the disclosure. Further, Formula Ib and Formula IB have been used interchangeably throughout the disclosure.

The term “at least one antibiotic” refers to any compound which is an antimicrobial compound that are active against microbes. The antibiotic compounds may be selected from antibacterial, antifungal, anti-infective, or anti-viral compounds. In the present disclosure, a pharmaceutical composition may comprise the compounds of the Formula I with a pharmaceutically acceptable carrier, and in combination with at least one antibiotic. Some of the examples of antibiotic include but not limited to erythromycin, azithromycin, clarithromycin, quinolones (ciprofloxacin or levofloxacin); ß lactams(e.g. penicillins, amoxicillin or piperacillin); cephalosporins (e.g. ceftriaxone or ceftazidime); carbapenems, (e.g. meropenem or imipenem etc); aminoglycosides (e.g. gentamicin or tobramycin; or oxazolidinones); antifungal triazole (e.g. or amphotericin); antibodies, cytokines, bactericidal/permeability increasing protein (BPI) products; rifampicin, isoniazid, pyrazinamide, ethambutol, moxifloxacin gatifloxacin, streptomycin, azido thymidine, sulfamethoxazole, or trimethoprim.

A term once described, the same meaning applies for it, throughout the disclosure.

As discussed in the background section, many of the commonly occurring infectious diseases have become greatly resistant to the currently available antimicrobial therapies. This has led to the development of difficult-to-treat infections which poses a major challenge to the field of modern medicine. Moreover, the conventionally used antibiotics are also reported to have toxic side effects and often exhibit short term antibacterial activity. Thus, in view of the above-mentioned shortcomings, the development of new compounds with improved effectiveness against evolving antibacterial resistance mechanisms and reduced toxicity is highly desirable.

In light of above, the present disclosure relates to compounds that successfully overcome the drawbacks of the existing antimicrobial therapies by providing improved pharmacological properties. The present disclosure relates to pyrazino-thiazenone compound isomers with R chirality. Surprisingly, the specific chirality of the present compounds provides them with improved antimicrobial efficacy against a wide spectrum of gram-positive and gram-negative bacteria including S. aureus, E. coli, K. pneumoniae and A. baumannii with highly reduced minimum inhibitory concentration at less than 0.03 μg/mL. Moreover, the compounds of the present disclosure also show highly reduced genotoxicity with in-vitro nucleus forming concentration greater than 120 μM which further makes them a safer alternative to treat infectious diseases caused by various microbes.

According to an embodiment, the present disclosure provides a compound selected from Formula Ia or Formula Ib, its stereoisomers, pharmaceutically acceptable salts, complexes, hydrates, solvates, tautomers, polymorphs, or pharmaceutically active derivatives thereof, wherein R₁ is selected from C₁₋₆ alkyl, C₂₋₆ alkenyl, C₃₋₆ cycloalkyl, CD₃, C₁₋₆ alkoxy, C₁₋₆ haloalkyl, or C₁₋₆ haloalkoxy; R₂ is selected from hydrogen, C₁₋₆ alkyl, halogen, hydroxy, or amino; R₃ is selected from hydrogen, halogen, hydroxyl, amino, cyano, C₁₋₆ alkoxy, C₁₋₆ haloalkyl, C₁₋₆ haloalkoxy, C₁₋₆ alkyl, NH—R₄, or —CH₂CH₂OH; R₄ is selected from C₁₋₆ alkyl, C₃₋₆ cycloalkyl, C₁₋₆ alkoxy, C₁₋₆ haloalkyl, C₁₋₆ haloalkoxy —CH₂CH₂OH, or —CH₂CH₂NH₂; X₁ is N or CR₃; X₂ is CR₅, O, N, or NR₆ when X₃ is CH or CH₂; R₅ is selected from hydrogen, cyano, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; R₆ is selected from hydrogen, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; X₃ is N or NR₇ when X₂ is CH₂ or CR₅; R₇ is selected from hydrogen, or C₁₋₆ alkyl; Y is N or CR₈; and R₈ is selected from hydrogen, halogen, cyano, C₁₋₆ alkyl, C₁₋₆ alkoxy, or C₁₋₆ haloalkoxy.

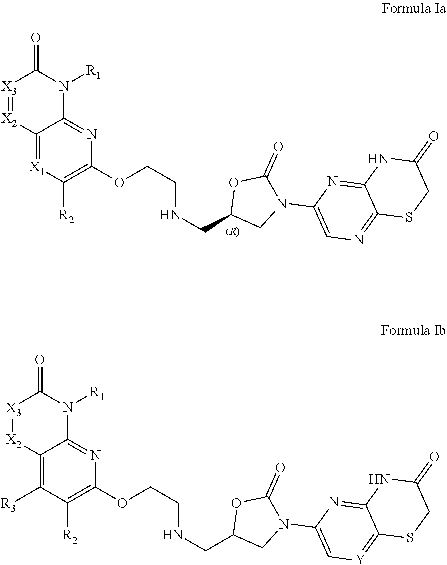

According to an embodiment, the present disclosure provides a compound selected from Formula Ia or Formula Ib, its stereoisomers, pharmaceutically acceptable salts, complexes, hydrates, solvates, tautomers, polymorphs, or pharmaceutically active derivatives thereof, wherein R₁ is selected from C₁₋₆ alkyl, C₂₋₆ alkenyl, CD₃, C₁₋₆ alkoxy, or C₁₋₆ haloalkyl; R₂ is selected from hydrogen, C₁₋₆ alkyl, or halogen; R₃ is selected from hydrogen, halogen, hydroxyl, C₁₋₆ alkoxy, C₁₋₆ alkyl, NH—R₄, or —CH₂CH₂OH; R₄ is selected from C₁₋₆ alkyl, C₃₋₆ cycloalkyl, C₁₋₆ alkoxy, C₁₋₆ haloalkyl, C₁₋₆ haloalkoxy —CH₂CH₂OH, or —CH₂CH₂NH₂; X₁ is N or CR₃; X₂ is CR₅, O, N, or NR₆ when X₃ is CH or CH₂; R₅ is selected from hydrogen, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; R₆ is selected from hydrogen, or C₁₋₆ alkyl, wherein C₁₋₆ alkyl is optionally substituted with one or more groups selected from hydroxyl, amino, or C₁₋₆ alkyl; X₃ is N or NR₇ when X₂ is CH₂ or CR₅; R₇ is selected from hydrogen, or C₁₋₆ alkyl; and Y is N or CR₈; and R₈ is selected from hydrogen, halogen, cyano, C₁₋₆ alkyl, or C₁₋₆ alkoxy.

According to an embodiment, the present disclosure provides a compound selected from Formula Ia or Formula Ib, its stereoisomers, pharmaceutically acceptable salts, complexes, hydrates, solvates, tautomers, polymorphs, or pharmaceutically active derivatives thereof, wherein R₁ is selected from C₁₋₆ alkyl, or CD₃; R₂ is hydrogen or halogen; R₃ is selected from hydrogen or C₁₋₆ alkyl; X₁ is N or CR₃; X₂ is CR₅, O, N, or NR₆ then X₃ is CH or CH₂; R₅ is selected from hydrogen, or C₁₋₆ alkyl, wherein C₁₋₆ alkyl is optionally substituted with one or more groups selected from hydroxyl, amino, or C₁₋₆ alkyl; R₆ is selected from hydrogen, or C₁₋₆ alkyl, wherein C₁₋₆ alkyl is optionally substituted with one or more groups selected from hydroxyl, amino, or C₁₋₆ alkyl; X₃ is N or NR₇ when X₂ is CH₂ or CR₅; R₇ is selected from hydrogen, or C₁₋₆ alkyl; Y is N or CR₈; and R₈ is selected from hydrogen, halogen, cyano, or C₁₋₆ alkyl.

According to an embodiment, the present disclosure provides a compound selected from Formula Ta or Formula Tb, its stereoisomers, pharmaceutically acceptable salts, complexes, hydrates, solvates, tautomers, polymorphs, or pharmaceutically active derivatives thereof, wherein R₁ is selected from C₁₋₆ alkyl, or CD₃; R₂ is hydrogen or halogen; R₃ is selected from hydrogen or C₁₋₆ alkyl; X₁ is N or CR₃; X₂ is CR₅, N, or NR₆ then X₃ is CH or CH₂; R₅ is selected from hydrogen, or C₁₋₆ alkyl; R₆ is selected from hydrogen, or C₁₋₆ alkyl; Y is N or CR₈; and R₈ is selected from hydrogen, halogen, cyano, or C₁₋₆ alkyl.

According to an embodiment, the present disclosure provides a compound of Formula Ta, its stereoisomers, pharmaceutically acceptable salts, complexes, hydrates, solvates, tautomers, polymorphs, or pharmaceutically active derivatives thereof, wherein R₁ is C₁₋₆ alkyl; R₂ is hydrogen; X₁ is N or CR₃; R₃ is hydrogen; X₂ is CR₅, or N then X₃ is CH or CH₂; R₅ is hydrogen.

According to an embodiment, the present disclosure provides a compound of Formula Ta, its pharmaceutically acceptable salts, complexes, hydrates, solvates, tautomers, polymorphs, or pharmaceutically active derivatives thereof, wherein R₁ is C₁₋₃ alkyl; R₂ is hydrogen; X₁ is N or CR₃; R₃ is hydrogen; X₂ is CR₅, or N then X₃ is CH; and R₅ is hydrogen.

According to an embodiment, the present disclosure provides a compound of Formula Ib, its stereoisomers, pharmaceutically acceptable salts, complexes, hydrates, solvates, tautomers, polymorphs, or pharmaceutically active derivatives thereof, wherein X₂ is NR₆ then X₃ is CH₂; R₆ is hydrogen. In another embodiment, the present disclosure provides a compound of Formula Ib, its stereoisomers, pharmaceutically acceptable salts, complexes, hydrates, solvates, tautomers, polymorphs, or pharmaceutically active derivatives thereof, wherein X₂ is N then X₃ is CH, wherein the double bond of X₃ will be inside the ring.

According to an embodiment, the present disclosure provides a compound of Formula Ib, its stereoisomers, pharmaceutically acceptable salts, complexes, hydrates, solvates, tautomers, polymorphs, or pharmaceutically active derivatives thereof, wherein R₁ is C₁₋₆ alkyl or CD₃; R₂ is hydrogen or halogen; R₃ is hydrogen; X₃ is NH or NR₇ then X₂ is CH₂ or CR₅; R₅ is hydrogen; Y is N.

According to an embodiment, the present disclosure provides a compound of Formula Ib, its stereoisomers, pharmaceutically acceptable salts, complexes, hydrates, solvates, tautomers, polymorphs, or pharmaceutically active derivatives thereof, wherein R₁ is C₁₋₆ alkyl or CD₃; R₂ is hydrogen or halogen; R₃ is hydrogen; X₂ is NH then X₂ is CH₂; and Y is N.

According to an embodiment, the present disclosure provides a compound of Formula Ia, its pharmaceutically acceptable salts, complexes, hydrates, solvates, tautomers, polymorphs, or pharmaceutically active derivatives thereof, wherein the compound is a) (R)-5-(((2-((4-methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one; and b) (R)-5-(((2-((5-methyl-6-oxo-5,6-dihydropyrido[2,3-b]pyrazin-3-yl) oxy) ethyl) amino) methyl)-3-(3-oxo-3,4-dihydro-2H-pyrazino[2,3-b][1,4]thiazin-6-yl)oxazolidin-2-one.

According to an embodiment, the present disclosure provides a compound of Formula Ib its pharmaceutically acceptable salts, complexes, hydrates, solvates, tautomers, polymorphs, or pharmaceutically active derivatives thereof, wherein the compound is selected from
(R)-6-(5-(((2-((4-methyl-3-oxo-1,2,3,4-tetrahydropyrido[2,3-b]pyrazin-6-yl) oxy) ethyl) amino)methyl)-2-oxooxazolidin-3-yl)-2H-pyrazino[2,3-b][1,4]oxazin-3 (4H)-one;
(S)-6-(5-(((2-((4-methyl-3-oxo-1,2,3,4-tetrahydropyrido[2,3-b]pyrazin-6-yl)oxy) ethyl) amino)methyl)-2-oxooxazolidin-3-yl)-2H-pyrazino[2,3-b][1,4]oxazin-3(4H)-one;
(R)-5-(((2-((7-fluoro-4-methyl-3-oxo-1,2,3,4-tetrahydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one;
(S)-5-(((2-((7-fluoro-4-methyl-3-oxo-1,2,3,4-tetrahydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one;
(R)-5-(((2-((4-(methyl-d3)-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl) oxazolidin-2-one; and
(S)-5-(((2-((4-(methyl-d3)-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl) oxazolidin-2-one.

According to an embodiment, the present disclosure provides a process of preparation of compound of Formula Ia, its pharmaceutically acceptable salts, complexes, hydrates, solvates, tautomers, polymorphs, or pharmaceutically active derivative thereof, said process comprising reacting Formula (X), and Formula (VI) in presence of at least one reducing agent to obtain the compounds of Formula Ia, wherein R₁ is selected from C₁₋₆ alkyl, C₂₋₆ alkenyl, C₃₋₆ cycloalkyl, CD₃, C₁₋₆ alkoxy, C₁₋₆ haloalkyl, or C₁₋₆ haloalkoxy; R₂ is selected from hydrogen, C₁₋₆ alkyl, halogen, hydroxy, or amino; R₃ is selected from hydrogen, halogen, hydroxyl, amino, cyano, C₁₋₆ alkoxy, C₁₋₆ haloalkyl, C₁₋₆ haloalkoxy, C₁₋₆ alkyl, NH—R₄, or —CH₂CH₂OH; R₄ is selected from C₁₋₆ alkyl, C₃₋₆ cycloalkyl, C₁₋₆ alkoxy, C₁₋₆ haloalkyl, C₁₋₆ haloalkoxy —CH₂CH₂OH, or —CH₂CH₂NH₂; X₁ is N or CR₃; X₂ is CR₅, O, N, or NR₆ when X₃ is CH or CH₂; R₅ is selected from hydrogen, cyano, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; R₆ is selected from hydrogen, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; X₃ is N or NR₇ when X₂ is CH₂ or CR₅; R₇ is selected from hydrogen, or C₁₋₆ alkyl; Y is N or CR₈; and R₈ is selected from hydrogen, halogen, cyano, C₁₋₆ alkyl, C₁₋₆ alkoxy, or C₁₋₆ haloalkoxy

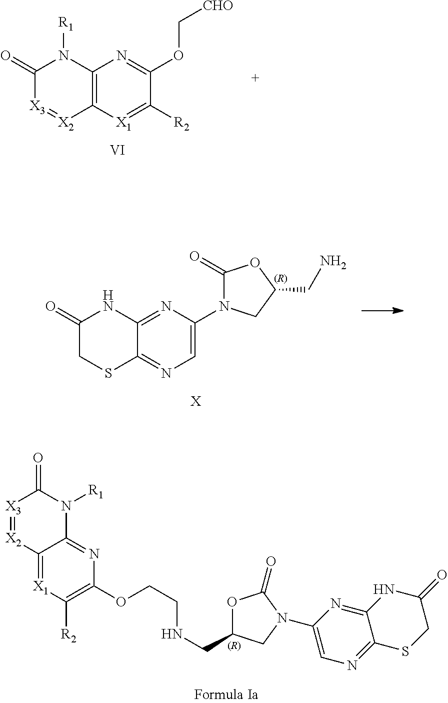

According to an embodiment, the present disclosure provides a process of preparation of compound of Formula Ib, its stereoisomers, pharmaceutically acceptable salts, complexes, hydrates, solvates, tautomers, polymorphs, or pharmaceutically active derivative thereof, said process comprising reacting Formula (VII), and Formula (XI) in presence of at least one reducing agent to obtain the compounds of Formula Ib, wherein R₁ is selected from C₁₋₆ alkyl, C₂₋₆ alkenyl, C₃₋₆ cycloalkyl, CD₃, C₁₋₆ alkoxy, C₁₋₆ haloalkyl, or C₁₋₆ haloalkoxy; R₂ is selected from hydrogen, C₁₋₆ alkyl, halogen, hydroxy, or amino; R₃ is selected from hydrogen, halogen, hydroxyl, amino, cyano, C₁₋₆ alkoxy, C₁₋₆ haloalkyl, C₁₋₆ haloalkoxy, C₁₋₆ alkyl, NH—R₄, or —CH₂CH₂OH; R₄ is selected from C₁₋₆ alkyl, C₃₋₆ cycloalkyl, C₁₋₆ alkoxy, C₁₋₆ haloalkyl, C₁₋₆ haloalkoxy —CH₂CH₂OH, or —CH₂CH₂NH₂; X₁ is N or CR₃; X₂ is CR₅, O, N, or NR₆ when X₃ is CH or CH₂; R₅ is selected from hydrogen, cyano, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; R₆ is selected from hydrogen, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl; X₃ is N or NR₇ when X₂ is CH₂ or CR₅; R₇ is selected from hydrogen, or C₁₋₆ alkyl; Y is N or CR₈; and R₈ is selected from hydrogen, halogen, cyano, C₁₋₆ alkyl, C₁₋₆ alkoxy, or C₁₋₆ haloalkoxy.

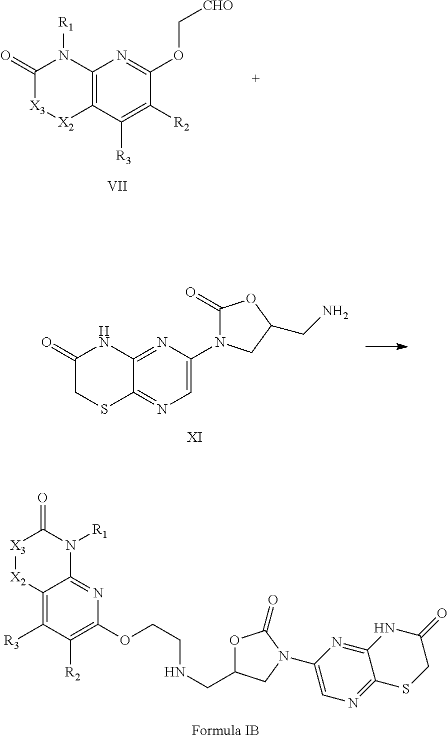

According to an embodiment, the present disclosure provides a process of preparation of compound of Formula Ia, its stereoisomers, pharmaceutically acceptable salts, complexes, hydrates, solvates, tautomers, polymorphs, or pharmaceutically active derivative thereof, said process comprising reacting Formula (X), and Formula (VI) in presence of at least one reducing agent selected from the group consisting of 2-picoline borane complex, sodium borohydride, sodium cyano borohydride, sodium triacetoxy borohydride, and combinations thereof to obtain the compounds of Formula Ia.

According to an embodiment, the present disclosure provides a process of preparation of compound of Formula Ib, its stereoisomers, pharmaceutically acceptable salts, complexes, hydrates, solvates, tautomers, polymorphs, or pharmaceutically active derivative thereof, said process comprising reacting Formula (VII), and Formula (XI) in presence of at least one reducing agent selected from the group consisting of 2-picoline borane complex, sodium borohydride, sodium cyano borohydride, sodium triacetoxy borohydride, and combinations thereof to obtain the compounds of Formula Ib.

According to an embodiment, the present disclosure provides a compound selected from Formula Ia or Formula Ib, its stereoisomers, pharmaceutically acceptable salts, complexes, hydrates, solvates, tautomers, polymorphs, or pharmaceutically active derivatives thereof, for use as a medicament.

According to an embodiment, the present disclosure provides a compound selected from Formula Ia or Formula Ib, its stereoisomers, pharmaceutically acceptable salts, complexes, hydrates, solvates, tautomers, polymorphs, or pharmaceutically active derivatives thereof, for use in killing or inhibiting the growth of a microorganism selected from the group consisting of bacteria, virus, fungi, and protozoa.

According to an embodiment, the present disclosure provides a compound selected from Formula Ta or Formula Tb, its stereoisomers, pharmaceutically acceptable salts, complexes, hydrates, solvates, tautomers, polymorphs, or pharmaceutically active derivatives thereof, for use in treatment of a bacterial infection caused by a gram-positive bacterium or a gram-negative bacterium.

According to an embodiment, the present disclosure provides the compound selected from Formula Ta or Formula Tb, its stereoisomers, pharmaceutically acceptable salts, complexes, hydrates, solvates, tautomers, polymorphs, or pharmaceutically active derivatives thereof, for use in treating a disease or condition in a patient wherein said disease or condition is caused by a microorganism selected from the group consisting of gram-positive, and gram-negative pathogens.

According to an embodiment, the present disclosure provides a pharmaceutical composition comprising a compound selected from Formula Ta or Formula Ib, its stereoisomers, pharmaceutically acceptable salts, complexes, hydrates, solvates, tautomers, polymorphs, or pharmaceutically active derivative thereof together with a pharmaceutically acceptable carrier.

According to an embodiment, the present disclosure provides a pharmaceutical composition comprising a compound selected from Formula Ta or Formula Ib, its pharmaceutically acceptable salts, complexes, hydrates, solvates, tautomers, polymorphs, or pharmaceutically active derivative thereof together with a pharmaceutically acceptable carrier, and in combination with at least one antibiotic.

According to an embodiment, the present disclosure provides a pharmaceutical composition comprising a compound selected from Formula Ia or Formula Tb, its pharmaceutically acceptable salts, complexes, hydrates, solvates, tautomers, polymorphs, or pharmaceutically active derivative thereof wherein the compound of Formula Ia or Formula Tb has enantiomeric excess in the range of 95 to 99.9%.

According to an embodiment, the present disclosure provides a pharmaceutical composition comprising a compound selected from Formula Ta or Formula Tb, its pharmaceutically acceptable salts, complexes, hydrates, solvates, tautomers, polymorphs, or pharmaceutically active derivative thereof wherein the compound of Formula Tb has enantiomeric excess in the range of 95 to 99.9% with respect to (R) isomeric form.

According to an embodiment, the present disclosure provides a pharmaceutical composition comprising a compound selected from Formula Ta or Formula Tb, its pharmaceutically acceptable salts, complexes, hydrates, solvates, tautomers, polymorphs, or pharmaceutically active derivative thereof wherein the compound of Formula Tb has enantiomeric excess in the range of 95 to 99.9% with respect to (S) isomeric form.

According to an embodiment, the present disclosure provides a use of compound selected from Formula Ta or Formula Tb, its stereoisomers, pharmaceutically acceptable salts, complexes, hydrates, solvates, tautomers, polymorphs, or pharmaceutically active derivative thereof, in killing or inhibiting the growth of a microorganism selected from the group consisting of bacteria, virus, fungi, and protozoa.

According to an embodiment, the present disclosure provides a use of compound selected from Formula Ta or Formula Tb, its stereoisomers, pharmaceutically acceptable salts, complexes, hydrates, solvates, tautomers, polymorphs, or pharmaceutically active derivative thereof, in killing or inhibiting the growth of bacteria.

According to an embodiment, the present disclosure provides a method for treatment of bacterial infection in a subject comprising: administering to the subject an effective amount of the compound selected from Formula Ia or Formula Ib as disclosed herein.

According to an embodiment, the present disclosure provides a method for treatment of bacterial infection in a subject comprising: administering to the subject an effective amount of the compound selected from Formula Ia or Formula Ib as disclosed herein, wherein the bacterial infection is caused by a gram-positive or a gram-negative pathogen.

According to an embodiment, the present disclosure provides a method for treatment of bacterial infection in a subject comprising: administering to the subject an effective amount of the compound selected from Formula Ia or Formula Ib as disclosed herein, wherein the bacterial infection is caused by E. coli, Pseudomonas aeruginosa, Klebsiella pneumoniae, Acinetobacter baumannii, Enterobacter cloacae, Staphylococcus aureus, Enterococcus faecalis Enterococcus faecium, Legionella pneumophila. Mycoplasma pneumonia, Acinetobacter haemolyticus Acinetobacter junii, Acinetobacter lwoffi, Burkholderia cepacia, Chlamydophila pneumoniae, Clostridium difficili, Enterobacter aerogenes, Enterobacter cloacae. Moraxella catarrhalis, Neisseria gonorrhoeae, Neisseria meningitides, Proteus mirabilis, Proteus houseri, Citrobacter freundii, Citrobacter kosari, Citrobacter barakii, Seratia marcescens, Klebsiella oxytoca, Morganella morganii, Helicobacter pyroli, or Mycobacterium tuberculosis.

The term “pharmaceutically acceptable” includes compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compound of Formula Ia or Formula Ib may form stable pharmaceutically acceptable acid or base salts, and in such cases administration of a compound as a salt may be appropriate. Examples of acid addition salts include acetate, adipate, ascorbate, benzoate, benzenesulfonate, bicarbonate, bisulfate, butyrate, camphorate, camphorsulfonate, choline, citrate, cyclohexyl sulfamate, diethylenediamine, ethanesulfonate, fumarate, glutamate, glycolate, hemisulfate, 2-hydroxyethylsulfonate, heptanoate, hexanoate, hydrochloride, hydrobromide, hydroiodide, hydroxymaleate, lactate, malate, maleate, methanesulfonate, meglumine, 2-naphthalenesulfonate, nitrate, oxalate, pamoate, persulfate, phenylacetate, phosphate, diphosphate, picrate, pivalate, propionate, quinate, salicylate, stearate, succinate, sulfamate, sulfanilate, sulfate, tartrate, tosylate (p-toluenesulfonate), trifluoroacetate, and undecanoate. Examples of base salts include ammonium salts; alkali metal salts such as sodium, lithium and potassium salts; alkaline earth metal salts such as aluminum, calcium and magnesium salts; salts with organic bases such as dicyclohexylamine salts and N10 methyl-D-glucamine; and salts with amino acids such as arginine, lysine, ornithine, and so forth. Also, basic nitrogen-containing groups may be quaternized with such agents as: lower alkyl halides, such as methyl, ethyl, propyl, and butyl halides; dialkyl sulfates such as dimethyl, diethyl, dibutyl; diamyl sulfates; long chain halides such as decyl, lauryl, myristyl and stearyl halides; arylalkyl halides such as benzyl bromide and others. Non-toxic physiologically acceptable salts are preferred, although other salts may be useful, such as in isolating or purifying the product.

The salts may be formed by conventional means, such as by reacting the free base form of the product with one or more equivalents of the appropriate acid in a solvent or medium in which the salt is insoluble, or in a solvent such as water, which is removed in vacuo or by freeze drying or by exchanging the anions of an existing salt for another anion on a suitable ion-exchange resin.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The present disclosure relates to a process of preparation of a composition comprising a compound of Formula Ia or Formula Ib its stereoisomers, pharmaceutically acceptable salts, complexes, hydrates, solvates, tautomers, polymorphs, or pharmaceutically active derivative thereof together with a carrier.

The present disclosure relates to a process of preparation of a pharmaceutical composition comprising a compound of Formula Ia or Formula Tb, its stereoisomers, pharmaceutically acceptable salts, complexes, hydrates, solvates, tautomers, polymorphs, or pharmaceutically active derivative thereof, together with a pharmaceutically acceptable carrier, optionally in combination with one or more other pharmaceutical compositions.

The compositions of the present disclosure may be obtained by conventional procedures using conventional pharmaceutical excipients well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate; granulating and disintegrating agents such as corn starch or alginic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents or procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or oil such as peanut oil, liquid paraffin, or olive oil.

Aqueous suspensions generally contain the active ingredient in finely powdered form or in the form of nano or micronized particles together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxyethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives such as ethyl or propyl p-hydroxybenzoate, anti-oxidants such as ascorbic acid, coloring agents, flavoring agents, and/or sweetening agents such as sucrose, saccharine or aspartame.

Oily suspensions may be formulated by suspending the active ingredient in a vegetable oil such as arachis oil, olive oil, sesame oil or coconut oil or in a mineral oil such as liquid paraffin. The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservative. Suitable dispersing or wetting agents and suspending agents are exemplified by those already mentioned above. Additional excipients such as sweetening, flavoring and coloring agents, may also be present.

The pharmaceutical compositions of the disclosure may also be in the form of oil-in-water emulsions. The oily phase may be a vegetable oil, such as olive oil or arachis oil, or a mineral oil, such as for example liquid paraffin or a mixture of any of these. Suitable emulsifying agents may be added, for example, naturally-occurring gums such as gum acacia or gum tragacanth, naturally-occurring phosphatides such as soya bean, lecithin, esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring or preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally acceptable diluent or solvent, for example a solution in 1,3-butanediol.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of the active ingredient.

Compositions for administration may also be formulated as a liposome preparation. The liposome preparation can comprise liposomes which penetrate the cells of interest or stratum corneum, and fuse with the cell membrane, resulting in delivery of the contents of the liposome into the cell. Other suitable formulations can employ niosomes. Niosomes are lipid vesicles similar to liposomes, with membrane consisting largely of nonionic lipids, some forms of which are effective for transporting compounds across the stratum corneum.

Compositions for administration may also be formulated as a depot preparation, which may be administered by implantation or by intramuscular injection. The compositions may be formulated with suitable polymeric or hydrophobic material (as an emulsion in acceptable oil), ion exchange resins, or sparingly soluble derivatives.

The compound of the present disclosure can also be administered in sustained release forms or from sustained release drug delivery systems.

For further information on formulation, drug delivery as well as processing techniques, the reader is referred to Remington's Pharmaceutical Sciences (21^{st} Edition, 2005, University of the sciences in Philadelphia, Lippincott William & Wilkins)

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 4 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes, the reader is referred to Chapter 25.3 in Volume 5 of Comprehensive Medicinal Chemistry (Corwin Hansch; Chairman of Editorial Board), Pergamon Press 1990 and Remington's Pharmaceutical Sciences (21 ^{st} Edition, 2005, University of the sciences in Philadelphia, Lippincott William & Wilkins).

As stated above, the size of the dosage required for the therapeutic or prophylactic treatment of a particular disease state will necessarily be varied depending on the host treated, the route of administration and the severity of the illness being treated. Preferably a daily dose in the range of 1-25 mg/kg is employed. Accordingly, the optimum dosage may be determined by the practitioner who is treating any particular patient.

In any of the pharmaceutical compositions, processes, methods, uses, medicaments, and manufacturing features mentioned herein, any of the alternate aspects of the compounds of the disclosure described herein also apply.

The compounds disclosed herein may be applied as a sole therapy or may involve, in addition to a compound of the disclosure, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination. Suitable classes and substances may be selected from one or more of the following: i) other antibacterial agents for example macrolides e.g. erythromycin, azithromycin or clarithromycin; quinolones e.g. ciprofloxacin or levofloxacin; B lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or ii) anti-infective agents for example, an antifungal triazole e.g. or amphotericin; and/or iii) biological protein therapeutics for example antibodies, cytokines, bactericidal/permeability increasing protein (BPI) products; and/or iv) one or more antibacterial agents useful in the treatment of Mycobacterium tuberculosis such as one or more of rifampicin, isoniazid, pyrazinamide, ethambutol, quinolones e.g. moxifloxacin or gatifloxacin, streptomycin; and/or v) efflux pump inhibitors.

According to an embodiment, the present disclosure relates to a compound of the Formula Ia or Formula Ib, or a pharmaceutically acceptable salt thereof and a chemotherapeutic agent selected from: i) one or more additional antibacterial agents; and/or ii) one or more anti-infective agents; and/or iii) biological protein therapeutics for example antibodies, cytokines, bactericidal/permeability increasing protein (BPI) products; iv) one or more antibacterial agents useful in the treatment of pulmonary tuberculosis, extra-pulmonary tuberculosis, avium infections, buruli ulcers; and/or v) one or more efflux pump inhibitors.

If not commercially available, the necessary starting materials for the procedures such as those described herein may be made by procedures which are selected from standard organic chemical techniques, techniques which are analogous to the synthesis of known, structurally similar compounds, or techniques which are analogous to the described procedure or the procedures described in the Examples.

It is noted that many of the starting materials for synthetic methods as described herein are commercially available and/or widely reported in the scientific literature, or could be made from commercially available compounds using adaptations of processes reported in the scientific literature. The reader is further referred to Advanced Organic Chemistry, 5th Edition, by Jerry March and Michael Smith, published by John Wiley & Sons 2001, for general guidance on reaction conditions and reagents.

It will also be appreciated that in some of the reactions mentioned herein, it may be necessary/desirable to protect any sensitive group in compounds. The instances where protection is necessary or desirable, and suitable methods for such protection are known to those skilled in the art. Conventional protecting groups may be used in accordance with standard practice (for illustration see T. W. Greene, Protective Groups in Organic Synthesis, published by John Wiley and Sons, 1991) and as described hereinabove.

### Abbreviations

The following abbreviations are employed in the examples and elsewhere herein:
TLC—thin layer chromatography;
HPLC—high pressure liquid chromatography;
NMR—nuclear magnetic resonance spectroscopy;
DMSO—dimethylsulfoxide;
MS—mass spectroscopy; ESP (or ES)—electrospray; EI—electron impact; APCI—atmospheric pressure chemical ionization;
THF—tetrahydrofuran;
DCM—dichloromethane;
MeOH—methanol;
DMF—dimethylformamide;
EtOAc—ethyl acetate;
LC/MS—liquid chromatography/mass spectrometry;
h—hour(s); min is minute(s);
d—day(s);
TFA—trifluoroacetic acid; v/v—ratio of volume/volume;
atm denotes atmospheric pressure;
rt denotes room temperature;
mg denotes milligram; g denotes gram;
μg denotes microgram;
μL denotes microliter;
mL denotes milliliter;
L denotes liter;
μM denotes micromolar;
mM denotes millimolar; M denotes molar;
N denotes normal; and
nm denotes nanometer.

### EXAMPLES

The following examples provide the details about the synthesis, activities and applications of the compounds of the present disclosure. It should be understood the following is representative only, and that the disclosure is not limited by the details set forth in these examples.

### Materials and Methods

Evaporations were carried out by rotary evaporation in vacuo and work up procedures were carried out after removal of residual solids by filtration; temperatures are quoted as ° C.; operations were carried out at room temperature, that is typically in the range 18 to 26° C. and without the exclusion of air unless otherwise stated, or unless the skilled person would otherwise work under an inert atmosphere; column chromatography (by the flash procedure) was used to purify compounds and was performed on Merck Kieselgel silica (Art. 9385) unless otherwise stated; in general, the course of reactions was followed by TLC, HPLC, or LC/MS and reaction times are given for illustration only; yields are given for illustration only and are not necessarily the maximum attainable; the structure of the end products of the disclosure was generally confirmed by NMR and mass spectral techniques. Proton magnetic resonance spectra were generally determined in DMSO d6 unless otherwise stated, using a Bruker DRX 300 spectrometer or a Bruker DRX-400 spectrometer, operating at a field strength of 300 MHz or 400 MHz, respectively. In cases where the NMR spectrum is complex, only diagnostic signals are reported. Chemical shifts are reported in parts per million downfield from tetramethylsilane as an external standard (δ scale) and peak multiplicities are shown thus: s, singlet; d, doublet; dd, doublet of doublets; dt, doublet of triplets; dm, doublet of multiplets; t, triplet; m, multiplet; br, broad. Fast atom bombardment (FAB) mass spectral data were generally obtained using a Platform spectrometer (supplied by Micromass) run in electrospray and, where appropriate, either positive ion data or negative ion data were collected or using Agilent 1100 series LC/MS equipped with Sedex 75ELSD, and where appropriate, either positive ion data or negative ion data were collected. The lowest mass major ion is reported for molecules where isotope splitting results in multiple mass spectral peaks (for example when chlorine is present). Reverse Phase HPLC was carried out using YMC Pack ODS AQ (100×20 mmID, S 5 Å particle size, 12 nm pore size) on Agilent instruments; each intermediate was purified to the standard required for the subsequent stage and was characterized in sufficient detail to confirm that the assigned structure was correct; purity was assessed by HPLC, TLC, or NMR and identity was determined by infrared spectroscopy (IR), mass spectroscopy or NMR spectroscopy as appropriate.

### Example 1

### General Process for the Preparation of the Compounds of Formula VI (a)

Compounds of Formula (II) were N-alkylated using alkyl halide to obtain the compounds of Formula (III). Further the compounds of Formula (III) were converted into compounds of Formula (IV) via aromatic nucleophilic substitution reaction using OHCH₂CH(OCH₂CH₃)₂, wherein Y is Cl or Br. Then, compounds of Formula (IV) were deprotected under acidic conditions to give compounds of Formula VI(a) as depicted in the general scheme below, wherein R₁ is selected from alkyl or ethyl, X₂ is substituted with substituents as disclosed in the embodiments above, or unsubstituted N, X₁ is selected from C or N.

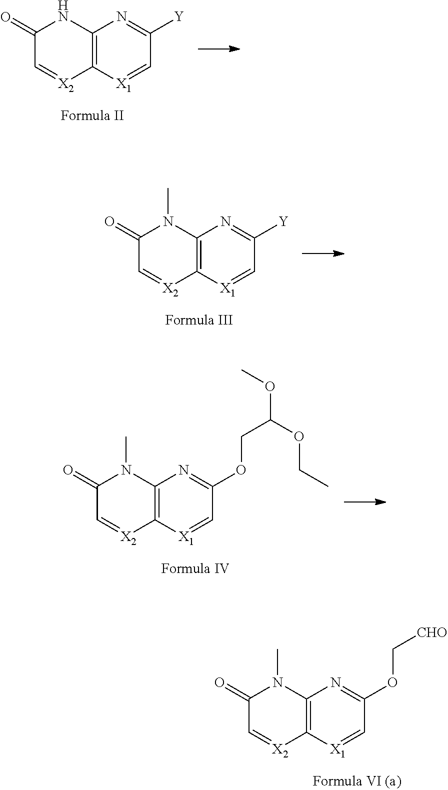

### General Process for the Preparation of the Compounds of Formula X

The compounds of Formula (X) were obtained from compounds of Formula (V) and compounds of Formula (Va) as summarised in the below Scheme. Palladium catalysed Buchwald coupling of compounds of formula (V) with Formula (VII) under optimal reaction conditions provided the compounds of formula (VIII). Further compounds of Formula (VIII) was converted to compounds of Formula (IX) via mesylation followed with azidation reaction. The reduction of azide functionality provided the compounds of Formula (X).

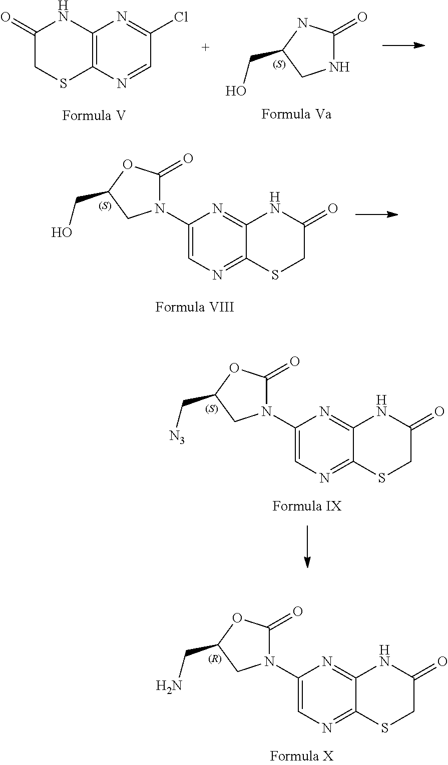

### Example 2

### General Process for the Preparation of the Compound of Formula Ia

The compound of Formula Ia can be prepared by reacting compounds of Formula (X) with compounds of Formula (VI) as shown in the below Scheme. The reductive amination of compounds Formula (X) with compounds of Formula (VI) provided the compound of Formula Ia.

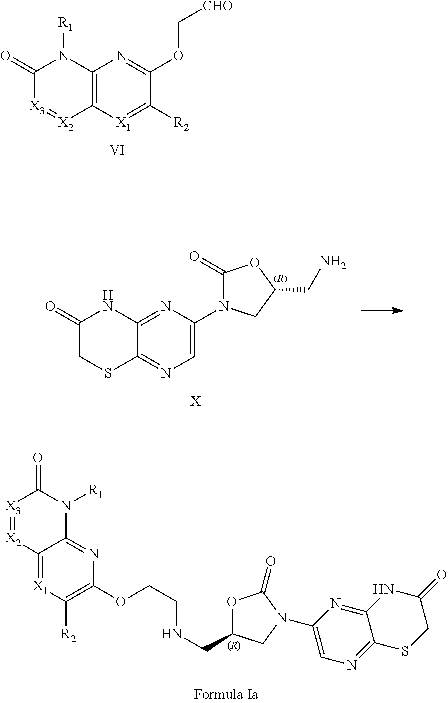

General process for the preparation of the compound of Formula Ib

The compound of Formula Ib can be prepared by reacting compounds of Formula (VII) with compounds of Formula (XI) as shown in the below Scheme. The reductive amination of compounds Formula (VII) with compounds of Formula (XI) provided the compound of Formula Ib.

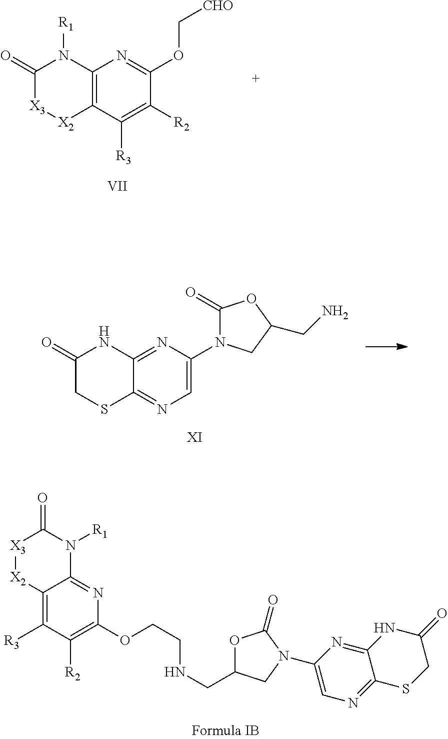

### Example 3

### Synthesis of Intermediates

### Synthesis of 2-((4-methyl-3-oxo-3,4-dihydropyrido[2,3-b]pyrazin-6-yl)oxy)acetaldehyde, Intermediate, VI

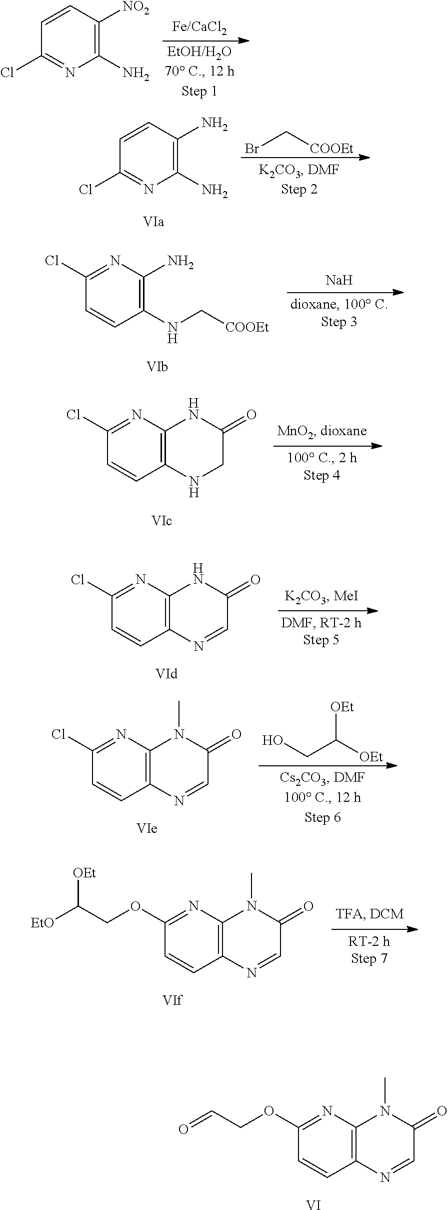

### Step-1: Synthesis of 6-chloropyridine-2,3-diamine (VIa)

To a stirred solution of 2-amino-3-nitro-6-chloropyridine (100 g, 0.576 mol) in a mixture of EtOH (1 L)/H₂O (1 L) were added CaCl2) (319.7 g, 2.880 mol) and Fe powder (321.79 g, 5.761 mol) successively at room temperature. The resulting mixture was heated at 100° C. for 6 h. After completion of the reaction, the reaction mixture was filtered through a celite bed. The filtrate was diluted with EtOAc (2 L) and washed with water (2×1 L). The organic layer was dried over sodium sulphate and concentrated in vacuo. The crude product was purified by column chromatography using silica gel (60-120 mesh) by eluting with 30% EtOAc in petroleum ether to afford pure product VIa as a brown solid. Yield: (59.8 g, 72.29%).

¹H NMR (400 MHz, DMSO-d₆): δ 6.69 (d, 1H, J=8 Hz), 6.65 (d, 1H, J=8 Hz), 5.78 (2H, s), 4.76 (2H, s). LC_MS Calc. for C₅H₆ClN₃ 143.57; Obs. 144.2 [M⁺+H]

### Step-2: Synthesis of ethyl (2-amino-6-chloropyridin-3-yl) glycinate (VIb)

To a stirred solution of VIa (100 g, 0.696 mol) in dry DMF (1 L) at room temperature under nitrogen atmosphere were successively added K₂CO₃ (134.8 g, 0.9756 mol) ethyl bromoacetate (164 g, 0.836 mol). The reaction mixture was heated at 100° C. for 6 h. After completion of the reaction, the reaction mixture was cooled to room temperature, diluted with EtOAc (2 L) and washed with water (2×800 mL). The combined organic layer was dried over sodium sulphate and concentrated to get the crude product VIb (99.2 g). The crude was taken to the next step without further purification

¹H NMR (400 MHz, DMSO-d₆): δ 6.5-6.4 (m, 2H), 5.9 (m, 2H), 5.39-5.35 (m, 1H), 4.16-4.08 (m, 2H), 3.94-3.91 (m, 2H), 1.2-1.17 (m, 4H) LC_MS: Calc. for C₉H₁₂C₁N₃O₂ 229.66; Obs. 230.2 [M⁺+H].

### Step-3: Synthesis of 6-chloro-1,4-dihydropyrido[2, 3-b]pyrazin-3(2H)-one (VIc)

To a stirred solution of VIb (100 g, 0.434 mol) in dry 1, 4 dioxane (500 mL) was added NaH (3.12 g, 0.13 mol) at room temperature under nitrogen atmosphere. The resulting mixture was heated at 100° C. for 1 h. After completion of the reaction, reaction mixture was cooled to room, temperature diluted with EtOAc (800 mL) and washed with water (2×500 mL). The organic layer was dried over sodium sulphate and concentrated in vacuo. The crude product was purified by column chromatography using silica gel (60-120 mesh) by eluting with 25% EtOAc in petroleum ether to afford pure product VIc as brown solid. Yield: (58.4, 72.87%). ¹H NMR (400 MHz, DMSO-d₆): δ 10.91 (brs, 1H), 6.98 (d, J=8.4 Hz, 1H), 6.83 (d, J=8 Hz, 1H), 6.33 (s, 1H), 3.82 (s, 2H). LC_MS: Calc. for C₇H₆ClN₃O: 183.60; Obs.: 184.1 [M⁺+H].

### Step-4: Synthesis of 6-chloropyrido [2, 3-b]pyrazin-3(4H)-one (VId)

To a stirred solution of VIc (50 g, 0.272 mol) in 1, 4-dioxane (500 mL) was added MnO₂ (142 g, 1.633 mol). The resulting mixture was heated at 100° C. for 6 h. After completion of the reaction, reaction mixture was filtered through a celite bed and washed with EtOAc (1000 mL). The filtrate was concentrated to get the crude product VId. The crude product was used for the next step without further purification. Yield: (42.3 g, 86%).

¹H NMR (400 MHz, DMSO-d₆): δ 13.10 (brs, 1H), 8.26-8.23 (m, 2H), 7.45-7.42 (m, 1H).

LC_MS Calc. for C₇H₄ClN₃O 181.58; Obs. 182.0 [M⁺+H].

### Step-5: Synthesis of 6-chloro-4-methylpyrido[2,3-b]pyrazin-3(4H)-one (VIe)

To a stirred solution of compound VId (30 g, 0.165 mol) in dry DMF (300 mL) at room temperature under nitrogen atmosphere was added potassium carbonate (45.74 g, 0.331 mol) and stirred for 10 mins. Then methyl iodide (94.13 g, 0.6629 mol) was added to the reaction mixture and stirred at room temperature for 1 h. After completion of the reaction, reaction mixture was poured into water (800 mL) and extracted with ethyl acetate (2×500 mL). The combined organic layer was separated, dried over sodium sulphate and concentrated in vacuo to get compound VIe as a brown solid. Yield: (23.4 g, 72.42%). ¹H NMR (400 MHz, DMSO-d₆) δ 8.33 (s, 1H), 8.30 (d, 1H, J=8 Hz), 7.52 (d, 1H, J=8 Hz), 3.58 (s, 3H). LC_MS Calc. for C₈H₆ClN₃O 195.61; Obs. 196.0 [M⁺+H].

### Step-6: Synthesis of 5-(2,2-diethoxyethyl)-4-methylpyrido[2,3-b]pyrazine-3, 6(4H,5H)-dione (VIf)

To a stirred solution of compound VIe (30 g, 0.153 mol) in DMF (300 mL) was added Cs₂CO₃ (124.92 g, 0.383 mol) at 0° C. under nitrogen atmosphere and stirred for 10 min. To this solution was added 2,2-Diethoxyethanol (30.8 g, 0.230 mol) and heated at 100° C. for 12 h. After completion of the reaction, reaction mixture was poured into water (400 mL) and extracted with ethyl acetate (2×500 mL). The combined organic layer was separated, dried over sodium sulphate and concentrated in vacuo to get the crude compound. It was purified by column chromatography on silica gel (60-120 mesh, 50% ethyl acetate in petroleum ether) to get compound VIf as a yellow solid. Yield: (25.3 g, 56.35%).

¹H NMR (400 MHz, DMSO-d₆): δ 8.14 (d, J=8.8 Hz, 1H), 8.12 (s, 1H), 6.87 (d, J=8.80 Hz, 1H), 4.91 (t, J=5.20 Hz, 1H), 4.38 (d, J=5.20 Hz, 2H), 3.72-3.70 (m, 2H), 3.68-3.56 (m, 5H), 1.16-1.13 (m, 6H). LC_MS Calc. for C₁₄H₁₉N₃O₄ 293.32; Obs. 294.2 [M⁺+H].

### Step-8: Synthesis of 2-(4-methyl-3,6-dioxo-4,6-dihydropyrido[2,3-b]pyrazin-5(3H)-yl)acetaldehyde (VI)

To a stirred solution of VIf (5 g, 0.017 mol) in dichloromethane (25 mL) was added trifluoroacetic acid (40 mL) in dropwise at 0° C. The reaction mixture was warmed to room temperature and stirred for 2 hours. After completion of the reaction, reaction mixture was diluted with dichloromethane (100 mL), neutralized with saturated sodium carbonate solution (pH˜7) and extracted with dichloromethane (3×100 mL). The combined organic layer was dried over sodium sulphate and concentrated under vacuo to get the crude product VI. The crude was used for the next step without further purification. Yield: (2.79 g, crude). LC_MS Calc. for C₁₀H₉N₃O₃ 219.20; Obs. 220.0 [M⁺+H].

### Synthesis of 2-((5-methyl-6-oxo-5,6-dihydropyrido[2,3-b]pyrazin-3-yl)oxy)acetaldehyde, Intermediate VII

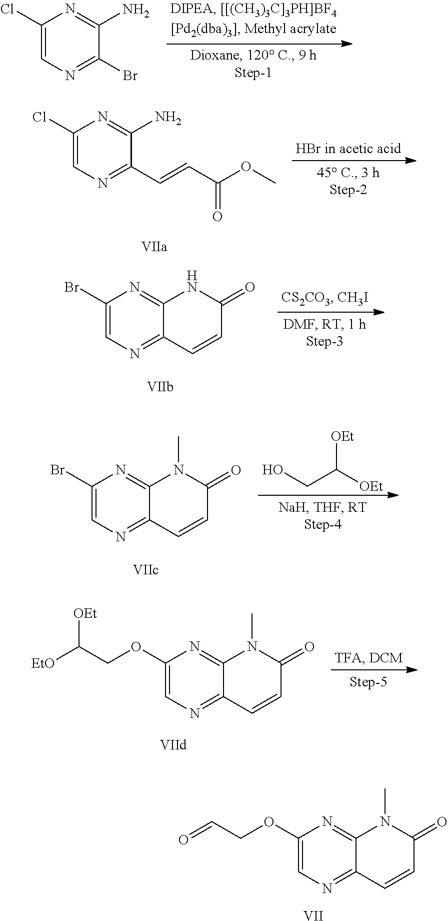

### Step-1: Synthesis of methyl (E)-3-(3-amino-5-chloropyrazin-2-yl) acrylate (VIIa)

To a stirred solution of 3-bromo-6-chloropyrazin-2-amine (40 g, 191 mmol) in dry 1,4-dioxane (400 mL) was added N, N-Diisopropylethylamine (53.3 mL, 305 mmol). The resulting mixture was degassed with a stream of nitrogen for 10 min. Then tris(dibenzylideneacetone)dipalladium(0) (3.50 g, 3.82 mmol), tri-tert-butylphosphonium tetrafluoroborate (2.77 g. 9.54 mmol) and methyl acrylate (34.23 mL, 382 mmol) were added successively. The resulting mixture was then heated at 120° C. for 9 hours. After completion of the reaction, reaction mixture was cooled to room temperature and a saturated aqueous solution of sodium bicarbonate (500 mL), and ethyl acetate (2 L) were added. The organic layer was separated and concentrated in vacuo to get the crude product, which was triturated with isopropyl ether (1 L). The solid precipitated was filtered and washed with isopropyl ether (500 mL), dried under vacuo to get compound VIIa as a yellow solid. Yield: (34 g, 82.95%). This compound was taken for next step without any further purification.

¹H NMR (400 MHz, DMSO-d₆): δ 7.91 (d, J=15.2, 1H), 7.86 (s, 1H), 7.32 (s, 2H), 6.76 (d, J=15.0 Hz, 1H), 3.74 (s, 3H). LC_MS Calc. for C8H8ClN3O2 213.62; Obs. 214.1 [M⁺+H]

### Step-2: Synthesis of 3-bromopyrido[2,3-b]pyrazin-6(5H)-one (VIIb)

A stirred solution of compound VIIa (34 g, 159.16 mmol) in HBr in acetic acid (33%, 510 mL, 15 vol) was added. The resulting mixture was then heated at 45° C. for 3 hours. After completion of the reaction, reaction mixture was cooled to room temperature and a saturated aqueous solution of sodium bicarbonate (400 mL), and ethyl acetate (2 L) were added. The organic layer was separated, dried over sodium sulphate and concentrated in vacuo to get compound VIIb as a yellow solid. Yield: (24 g, 66.70%).

¹H NMR (400 MHz, DMSO-d₆): δ 12.71 (s, 1H), 8.66 (s, 1H), 7.99-7.93 (m, 1H), 6.86 (d, J=9.6 Hz, 1H). LC_MS Calc. for C7H4BrN3O 226.03; Obs. 227.8 [M++H].

### Step-3: Synthesis of 3-bromo-5-methylpyrido[2,3-b]pyrazin-6(5H)-one (VIIc)

To a stirred solution of compound VIIb (18 g, 79.63 mmol) in dry DMF (180 mL) at room temperature under nitrogen atmosphere was added Cs2CO3 (51.89 g, 159.27 mmol) and stirred for 10 min. Then methyl iodide (45.21 g, 318.54 mmol) was added at once. The reaction mixture was stirred at RT for 1 h. After completion of the reaction, reaction mixture was poured into water (400 mL) and extracted with ethyl acetate (2×600 mL). The combined organic phase was separated, dried over sodium sulphate and concentrated in vacuo to get compound VIIc as a brown solid. Yield: (13.8 g, 72.25%).

¹H NMR (400 MHz, DMSO-d₆): δ 8.73 (s, 1H), 8.02 (d, J=10.0 Hz, 1H), 7.02 (d, J=10.0 Hz, 1H), 3.06 (s, 3H). LC_MS Calc. for C8H6BrN3O 240.06; Obs. 240.1 [M++H].

### Step-4: Synthesis 3-(2,2-diethoxyethoxy)-5-methylpyrido[2,3-b]pyrazine-6(5H)-one (VIId)

To a stirred solution of compound VIIc (13.8 g, 57.48 mmol) in THF (130 mL) at 0° C. under nitrogen atmosphere was added 2,2-diethoxyethanol (9.25 g, 68.98 mmol). To this solution, sodium hydride (1.38 g, 57.48 mmol) was added cautiously in portions over a period of 10 minutes. The reaction mixture was then stirred at room temperature for 30 min. After completion of the reaction, reaction mixture was poured into water (200 mL) and extracted with ethyl acetate (1 L). The organic layer was separated, dried over sodium sulphate and concentrated in vacuo to get the crude compound XIII. It was purified by column chromatography on silica gel (60-120 mesh, 50% ethyl acetate in petroleum ether) to afford the compound VIId as a yellow liquid. Yield: (9.5 g, 56.35%).

¹H NMR (400 MHz, DMSO-d₆): δ 8.25 (s, 1H), 7.94 (d, J=9.60 Hz, 1H), 6.73 (d, J=9.9 Hz, 1H), 4.92 (d, J=4.8 Hz, 1H), 4.42-4.41 (m, 2H), 3.73-3.70 (m, 1H), 3.61-3.44 (m, 6H). 1.22-1.11 (m, 6H). LC_MS Calc. for C14H19N3O4 293.32; Obs. 294.2 [M⁺+H].

### Step-5: Synthesis of 2-((5-methyl-6-oxo-5,6-dihydropyrido[2,3-b]pyrazine-3-yl) oxy) acetaldehyde (VII)

To a stirred solution of VIId (7.8 g, 26.83 mmol) in dichloromethane (78 mL, 10 vol) at 0° C. was added trifluoroacetic acid (62.4 mL, 8 vol) in dropwise. The reaction mixture was warmed to room temperature and stirred for 2 hours. After completion of the reaction, the reaction mixture was quenched with ice water, neutralized with saturated sodium bicarbonate solution (pH˜7) and extracted with dichloromethane (2×500 mL). The combined organic layer was dried over sodium sulphate and concentrated to get crude product VII as a yellow gummy material. The crude product was taken for the next step without further purification. Yield: (4.5 g, 77.32%) LC_MS Calc. for C10H9N3O3 219.20; Obs. 220.1 [M⁺+H].

### Synthesis of 6-chloro-2H-pyrazino[2,3-b][1,4]thiazin-3(4H)-one, Intermediate V

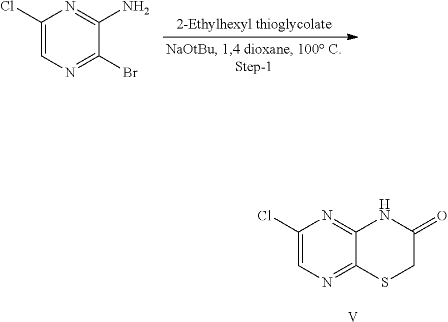

To a stirred solution of 3-bromo-6-chloropyrazin-2-amine (100 g, 0.479 mol) in 1, 4-dioxane (1.5 L) at room temperature under nitrogen atmosphere was added sodium tert-butoxide (138.3 g, 1.439 mol) and stirred for 30 minutes. Then 2-ethylhexylthioglycolate (156.8 g, 0.7676 mol) was added in dropwise over a period of 30 minutes at room temperature. The resulting mixture was heated at 100° C. for 2 hours. The progress of the reaction was monitored by TLC. After completion of the reaction, reaction mixture was cooled to room temperature and concentrated under reduced pressure to remove the 1,4-dioxane. The residue obtained was diluted with water (750 mL) and neutralized by HCl (1.5 N). The precipitated solid was filtered out and dried to get compound V as an off white solid. Yield: (78 g, 80.64%)

¹H-NMR (400 MHz, DMSO-d₆): δ 11.54 (s, 1H), 8.25 (s, 1H), 3.83 (s, 2H). LC_MS: Calc. for C₆H₄ClN₃O_{S}: 201.63; Obs.: 199.9 [MM+1H].

### Synthesis of (R)-5-(aminomethyl)-3-(3-oxo-3,4-dihydro-2H-pyrazino[2,3-b][1,4]thiazin-6-yl)oxazolidin-2-one, Intermediate X

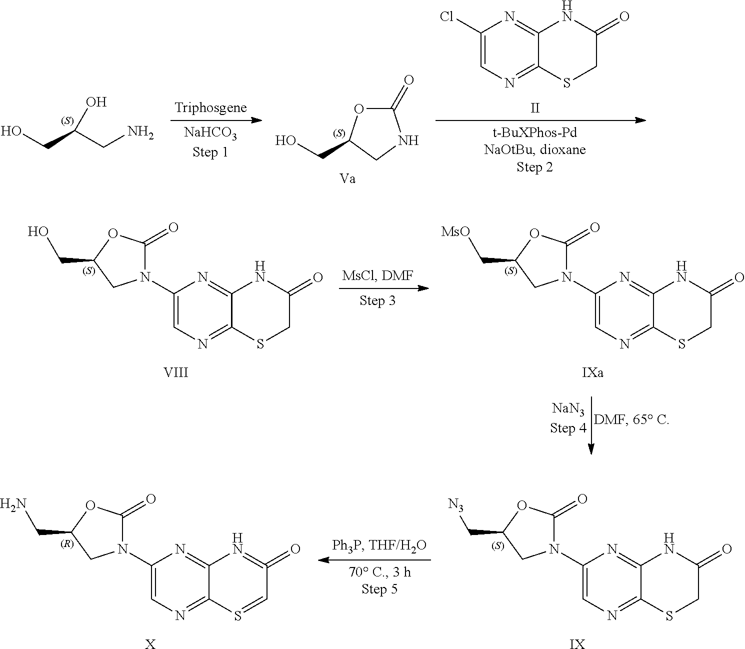

### Step-1: Synthesis of (S)-5-(hydroxymethyl)oxazolidin-2-one (Va)

To a stirred solution of amino diol (100 g, 1.097 mol), NaHCO₃ (276.5 g, 3.292 mol) in H₂O (1 L), cooled to 0° C., was added triphosgene (97.7 g, 0.329 mmol). The reaction mixture was warmed to room temperature and stirred for 16 h. Then reaction mixture was neutralized with aqueous HCl (4 N, pH˜7) and concentrated in vacuo. The crude product was purified by column chromatography on silica gel (60-120 mesh, 10% MeOH in CH₂Cl₂) to afford Va as a white solid. Yield: (70 g, 54.49%).

¹H NMR (400 MHz, DMSO-d₆): δ 7.38 (s, 1H), 5.08 (brs, 1H), 4.56-4.50 (m, 1H), 3.55-3.52 (m, 1H), 3.47-3.43 (m, 2H), 3.24-3.16 (m, 1H). LC_MS: Calc. for C₄H₇NO₃: 117.10; Obs.: 118.2 [M⁺+H].

### Step-2: Synthesis of (S)-5-(hydroxymethyl)-3-(3-oxo-3,4-dihydro-2H-pyrazino[2,3-b][1,4]thiazin-6-yl)oxazolidin-2-one (VIII)

To a stirred solution of compound V (100 g, 0.4960 mol) and (S)-5-(hydroxymethyl) oxazolidin-2-one Va (CAS: 97859-51-3, 58.03 g, 0.4960 mol) in 1.4-dioxane (1.5 L) was added sodium tert-butoxide (71.5 g, 0.7440 mol) at room temperature. The resulting mixture was degassed with a stream of nitrogen for 10 minutes. Then t-Butyl-X-Phos Palladacycle (19.69 g, 0.0248 mol) was added at room temperature and again degassed with nitrogen for 5 minutes. The resulting mixture was then heated at 100° C. for 5 hours. After completion of the reaction, reaction mixture was cooled to room temperature and concentrated in vacuo. The residue obtained was diluted with water (500 mL), neutralized with aqueous HCl (1.5 N, pH˜7). The solid precipitated out was filtered and washed with diethyl ether, dried under vacuo to get compound VIII as a brown solid. Yield: (100 g, crude). The crude was taken to next step without any further purification.

¹H NMR (300 MHz, DMSO-d₆): δ 11.23 (s, 1H), 8.81 (s, 1H), 5.24-5.07 (m, 1H), 4.77-4.70 (m, 1H), 4.08 (t, J=11.2 Hz, 1H), 3.89-3.87 (m, 1H), 3.76-3.72 (m, 3H), 3.59-3.57 (m, 1H). LC_MS: Calc. for C₁₀H₁₀N₄O₄S: 282.27; Obs.: 283 [M⁺+H].

### Step-3: Synthesis of (S)-(2-oxo-3-(3-oxo-3,4-dihydro-2H-pyrazino[2,3-b][1,4]thiazin-6-yl)oxazolidin-5-yl)methyl methanesulfonate (IXa)

To a stirred solution of VIII (100 g, 0.3542 mol) in dry DMF (700 mL) at 0° C. under nitrogen atmosphere were added triethylamine (148 mL, 1.0628 mol) and methane sulfonyl chloride (41.1 mL, 0.5314 mol) successively. The reaction mixture was then warmed to room temperature and stirred for 2 h. After completion of the reaction, reaction mixture was quenched with water, the solid formed was filtered, washed with petroleum ether and dried to get compound IXa as a brown solid (85 g, crude). The crude product was taken to next step without further purification.

LC_MS: Calc. for C11H12N4O6S2: 360.36; Obs.: 361.00 [M⁺+H].

### Step-4: Synthesis of (S)-5-(azidomethyl)-3-(3-oxo-3,4-dihydro-2H-pyrazino[2,3-b][1,4]thiazin-6-yl)oxazolidin-2-one (IX)

To a stirred solution of IXa (50 g, 0.1387 mol) in DMF (350 mL) at 0° C. under nitrogen atmosphere was added sodium azide (2.16 g, 0.4162 mol). The reaction mixture was then heated at 65° C. and stirred for 3 h. After completion of the reaction, reaction mixture was quenched with water, the solid precipitated was filtered, washed with petroleum ether and dried to get compound IXa as a brown solid (26 g, 60.80%).

¹H NMR (400 MHz, DMSO-d₆): δ 11.25 (s, 1H), 8.80 (s, 1H), 4.95 (brs, 1H), 4.15 (t, J 12.80 Hz, 1H), 3.85-3.70 (m, 5H). LC_MS: Calc. for C10H9N7O3S: 307.29; Obs.: 308 [M⁺+H].

### Step-5: Synthesis of (R)-5-(aminomethyl)-3-(3-oxo-3,4-dihydro-2H-pyrazino[2,3-b][1,4]thiazin-6-yl)oxazolidin-2-one (X)

To a stirred solution of compound IX (50 g, 0.1628 mol) in a mixture of THF: H₂O (1:1) (800 mL) under nitrogen atmosphere was added PPh3 (128 g, 0.488 mol) at room temperature. The reaction mixture was heated at 70° C. for 3 hours. After completion of the reaction by TLC, the reaction mixture was cooled to room temperature and extracted with ethyl acetate (2×500 mL). The aqueous layer was separated and concentrated in vacuo to get compound X (28 g, 61.18%).

¹H NMR (400 MHz, DMSO-d₆): δ8.80 (s, 1H), 4.69 (s, 2H), 4.08-4.05 (m, 1H), 3.86-3.76 (m, 4H), 2.85-2.73 (m, 3H). LC_MS: Calc. for C10H11N5O3S: 281.29; Obs.: 282.1 [M⁺+H].

### Synthesis of (R)-5-(((2-((4-(methyl-d3)-3-oxo-3,4-dihydropyrido[2,3-b]pyrazin-6-yl)oxy) ethyl)amino)methyl)-3-(3-oxo-3,4-dihydro-2H-pyrazino[2,3-b][1,4]thiazin-6-yl)oxazolidin-2-one. Intermediate XI

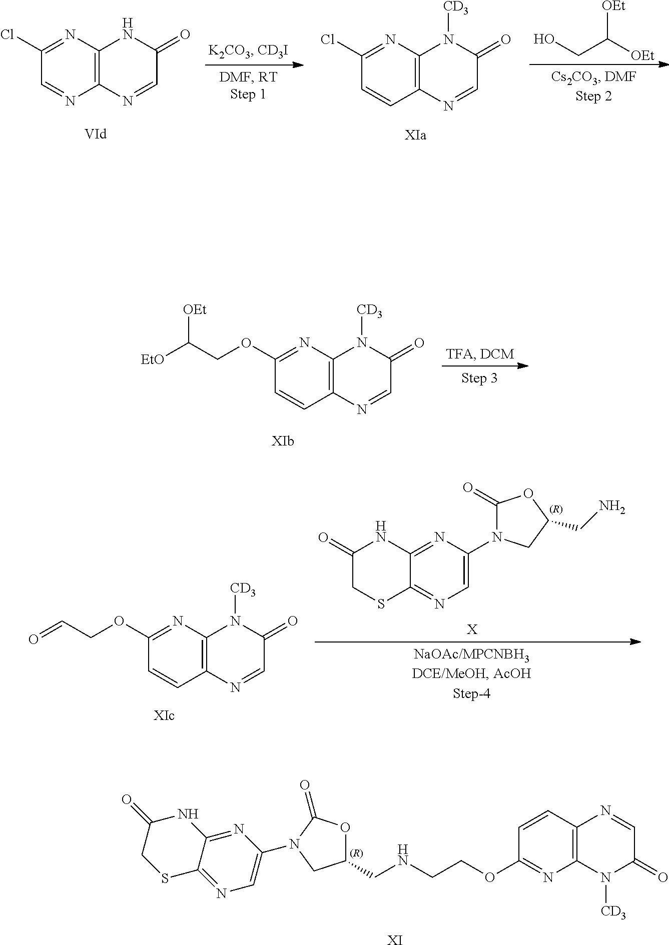

### Step-1: Synthesis of 6-chloro-4-(methyl-d3) pyrido[2,3-b]pyrazin-3(4H)-one (XIa)

To a stirred solution of compound VId (5 g, 0.02754 mol) in dry DMF (50 mL), was added potassium carbonate (7.6 g, 0.0550 mol) at room temperature. Then iodomethane-d₃ (15.97 g, 0.1101 mol) was added at room temperature. The reaction mixture was then stirred at room temperature for 1 h. After completion of the reaction, reaction mixture was poured into water (50 mL) and extracted with ethyl acetate (2×200 ml). The combined organic layer was separated, dried over sodium sulphate and concentrated in vacuo to get crude as a brown solid. The crude product was purified by column chromatography using silica gel (60-120 mesh) by eluting with 15% EtOAc in petroleum ether to afford pure product XIa as brown solid. Yield: (3.9 g, 72.25%).

¹H NMR (400 MHz, DMSO-d₆): δ 8.32 (s, 1H), 8.30 (d, J=10.8 Hz, 1H), 7.51 (d, J=11.20 Hz, 1H). LC_MS Calc. for C₈H₃D₃ClN₃O: 198.62; Obs.: 199.0 [M⁺+H].

### Step-2: Synthesis of 6-(2,2-diethoxyethoxy)-4-(methyl-d₃)pyrido[2,3-b]pyrazin-3(4H)-one (XIb)

To a mixture of XIa (3 g, 0.015 mol) in DMF (20 mL) at room temperature under nitrogen atmosphere were added Cs₂CO₃ (12.3 g, 0.037 mol) and 2,2-diethoxyethanol (6 g, 0.045 mol) successively. The resulting mixture was stirred at 80° C. for 2 hours. The reaction mixture was cooled and quenched with water extracted with ethyl acetate (2×125 mL). The combined organic layer was washed with brine (2×50 mL), dried over Na₂SO₄ and concentrated under reduced pressure. The crude product was purified by column chromatography using silica gel (230-400 mesh) eluting with 15% of ethyl acetate in petroleum ether to get compound XIb (3.1 g, 77.5%).

¹H NMR (400 MHz, DMSO-d₆): δ 8.16-8.13 (m, 2H), 6.87 (d, J=11.20 Hz, 1H), 4.92 (t, J=6 Hz, 1H), 4.38 (d, J=6.4 Hz, 2H), 3.72-3.58 (m, 4H), 1.16-1.12 (m, 6H).

LC_MS Calc. for C₁₄H₁₆D₃N₃O₄: 296.34; Obs.: 297.1 [M⁺+H].

### Step-3: Synthesis of 2-((4-(methyl-d3)-3-oxo-3,4-dihydropyrido[2,3-b]pyrazin-6-yl)oxy) acetaldehyde (XIc)

To a stirred solution of XIb (1 g, 0.0033 mol) in dichloromethane (3 mL) was added trifluoroacetic acid (8 mL) in dropwise at 0° C. The reaction mixture was warmed to room temperature and stirred for 2 hours. After completion of the reaction, reaction mixture was diluted with dichloromethane (50 mL), neutralised with saturated sodium carbonate solution (pH˜7) and extracted with dichloromethane (3×100 ml). The combined organic layer was dried over sodium sulphate and concentrated in vacuo to get crude product XIc. The crude product obtained was used for the next step without further purification. Yield: (0.6 g, crude). LC_MS Calc. for C₁₀H₆D₃N₃O₃: 222.21; Obs.: 223.1 [M⁺+H].

### Step-4: (R)-5-(((2-((4-(methyl-d3)-3-oxo-3,4-dihydropyrido[2,3-b]pyrazin-6-yl)oxy) ethyl)amino)methyl)-3-(3-oxo-3,4-dihydro-2H-pyrazino[2,3-b][1,4]thiazin-6-yl)oxazolidin-2-one (XI)

To a mixture of X (0.9 g, 4 mmol) and XIc (1 g, 4 mmol) in a dry MeOH (100 mL)/DCE (100 mL) were added AcOH (1 mL), sodium acetate (2 g, 0.02 mol) and MPCNBH₃ resin (1 g) at room temperature under nitrogen atmosphere. The resulting mixture was continued to stir at room temperature for 3 hours. After completion of the reaction mixture, reaction mixture was quenched with water (5 mL) and concentrated in vacuo. The crude product was purified by column chromatography using silica gel (230-400 mesh) eluting with 6% methanol in dichloromethane to afford the XI as a yellow solid (0.51 g, 25.5%).

¹H NMR (300 MHz, DMSO-d₆): δ 11.21 (brs, 1H), 8.76 (s, 1H), 8.14 (s, 1H), 8.10-8.07 (m, 1H), 6.80 (d, J=8.7 Hz, 1H), 4.85-4.83 (m, 1H), 4.48-4.42 (m, 2H), 4.12-4.06 (m, 1H), 3.85-3.80 (m, 1H), 3.76 (s, 2H), 3.23 (s, 1H), 3.03-2.97 (m, 4H).

LC_MS: Calc. for C₂₀H₁₇D₃N₈O₅S: 487.50; Obs.: 488.0 [M⁺+H]. HPLC Purity=97.93%, X-Bridge C8 (50×4.6) mm, 5 μm, Mobile Phase A: 0.1% TFA in water, Mobile Phase B: Acetonitrile.

### Synthesis of (S)-5-(((2-((4-(methyl-d3)-3-oxo-3,4-dihydropyrido[2,3-b]pyrazin-6-yl)oxy) ethyl)amino)methyl)-3-(3-oxo-3,4-dihydro-2H-pyrazino[2,3-b][1,4]thiazin-6-yl)oxazolidin-2-one. Intermediate XII

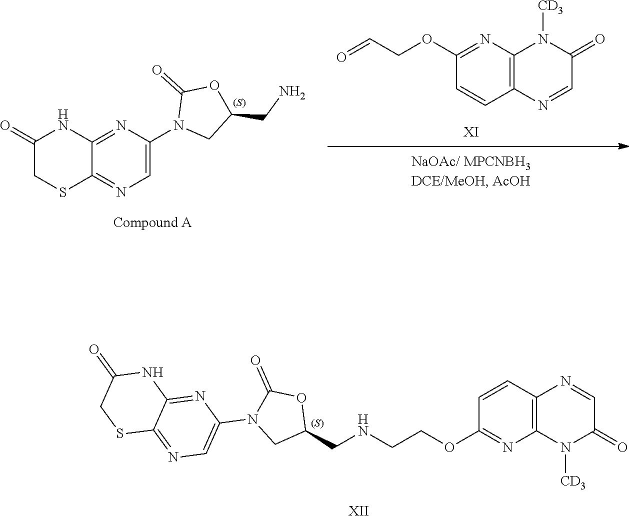

To a mixture of compound A (WO2019186590, 0.9 g, 4 mmol) and XI (1 g, 4 mmol) in a dry MeOH (100 mL)/DCE (100 mL) were added AcOH (1 mL), sodium acetate (2 g, 0.02 mol) and MPCNBH₃ resin (1 g) at room temperature under nitrogen atmosphere. The resulting mixture was continued to stir at room temperature for 3 hours. After completion of the reaction mixture, reaction mixture was quenched with water (5 mL) and concentrated in vacuo. The crude product was purified by column chromatography using silica gel (230-400 mesh) eluting with 6% methanol in dichloromethane to afford the XII as a yellow solid (0.42 g, 21.27%).

¹H NMR (300 MHz, DMSO-d₆): δ 11.21 (brs, 1H), 8.76 (s, 1H), 8.14 (s, 1H), 8.10-8.07 (m, 1H), 6.80 (d, J=8.7 Hz, 1H), 4.85-4.83 (m, 1H), 4.48-4.42 (m, 2H), 4.12-4.06 (m, 1H), 3.85-3.80 (m, 1H), 3.76 (s, 2H), 3.23 (s, 1H), 3.03-2.97 (m, 4H). LC_MS: Calc. for C₂₀H₁₇D₃N₈O₅S: 487.50; Obs.: 488.0 [M⁺+H]. HPLC Purity=97.93%, X-Bridge C8 (50×4.6) mm, 5 m, Mobile Phase A: 0.1% TFA in water, Mobile Phase B: Acetonitrile.

### Synthesis of (R)-5-(((2-((7-fluoro-4-methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one. Intermediate XIII

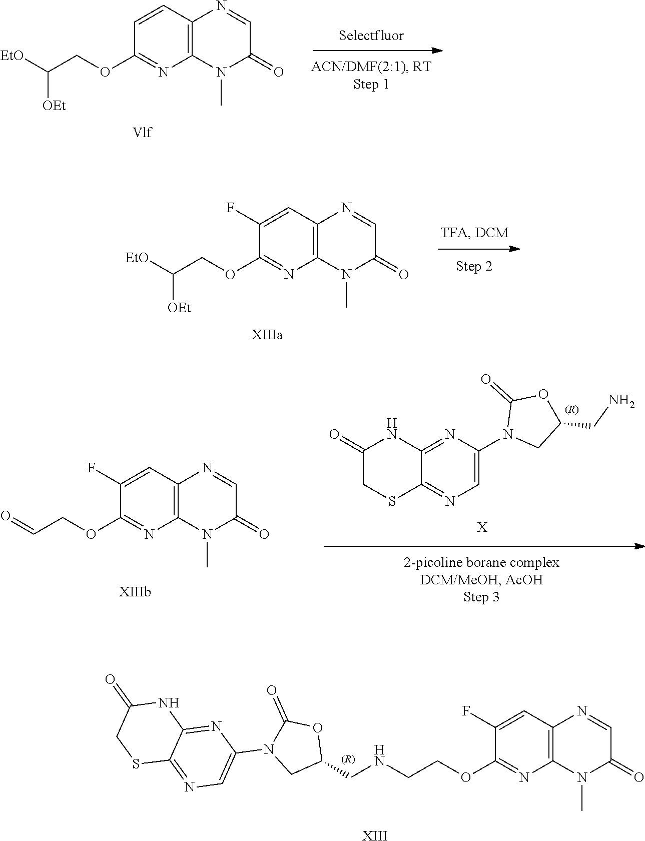

### Step-1: 6-(2,2-diethoxyethoxy)-7-fluoro-4-methylpyrido[2,3-b]pyrazin-3(4H)-one (XIIIa)

To a stirred solution of VIf (6 g, 20.45 mmol) in a mixture of CH₃CN/DMF (60 mL, 2:1) at 0° C. under nitrogen atmosphere was added selectfluor (21.73 g, 61.36 mmol). The resulting mixture was stirred at room temperature for 48 h.

After completion of the reaction, reaction mixture was concentrated in vacuo. The crude product obtained was purified by column chromatography by eluting with 28% ethyl acetate in petroleum ether to afford compound XIIIa as a pale-yellow viscous liquid. Yield: (1.5 g, 23.58%). LC_MS Calc. for C₁₄H₁₈FN₃O₄: 311.31; Obs.: 312.2 [M⁺+H].

### Step-2: 2-((7-fluoro-4-methyl-3-oxo-3,4-dihydropyrido[2,3-b]pyrazin-6-yl)oxy) acetaldehyde. (XIIIb)

To a stirred solution of XIIIa (1.5 g, 4.81 mmol) in dichloromethane (10 mL) was added trifluoroacetic acid (12 mL) in dropwise at 0° C. The reaction mixture was warmed to room temperature and stirred for 2 hours. After completion of the reaction, the reaction mixture was diluted with dichloromethane (30 mL) and neutralized with saturated aqueous sodium bicarbonate solution to pH˜7 and brine (30 mL). The organic layer was dried over sodium sulphate and concentrated in vacuo to get crude product XIIIb. The crude product was used for the next step without further purification. Yield: (0.95 g, crude). LC_MS Calc. for C₁₀H₈FN₃O₃: 237.19; Obs.: 238.2 [M⁺+H].

### Step-3: (R)-5-(((2-((7-fluoro-4-methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one. (XIII)

To a stirred mixture of XIIIb (0.5 g, 2.1 mmol) and X (0.65 g, 2.3 mmol) in a dry MeOH (40 mL)/DCM (40 mL) were added AcOH (0.5 mL) and 2-picoline borane complex (0.15 g, 1.47 mmol) at room temperature under nitrogen atmosphere. The resulting mixture was continued to stir at room temperature for 1 hour. After completion of the reaction mixture, reaction mixture was quenched with water (10 mL) and concentrated in vacuo. The obtained crude product was purified by PREP HPLC to get XIII as a formate salt (off-white solid). Yield: (0.15 g, 14.15%).

¹H NMR (300 MHz, DMSO-d₆): δ 11.19 (brs, 1H), 8.75 (s, 1H), 8.19-8.15 (m, 3H), 4.82-4.80 (m, 1H), 4.61-4.55 (m, 2H), 4.07-4.04 (m, 1H), 3.84-3.80 (m, 3H), 3.60 (s, 3H), 3.04 (t, J=10.5 Hz, 2H), 2.99-2.95 (m, 2H). LC_MS: Calc. for C₂₀H₁₉FN₈O₅S: 502.48; Obs.: 503 [M⁺+H]. HPLC Purity=98.08%, X-Bridge C8 (50×4.6) mm, 5 μm, Mobile Phase A: 0.1% TFA in water, Mobile Phase B: Acetonitrile.

### Synthesis of (S)-5-(((2-((7-fluoro-4-methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one. Intermediate XIV

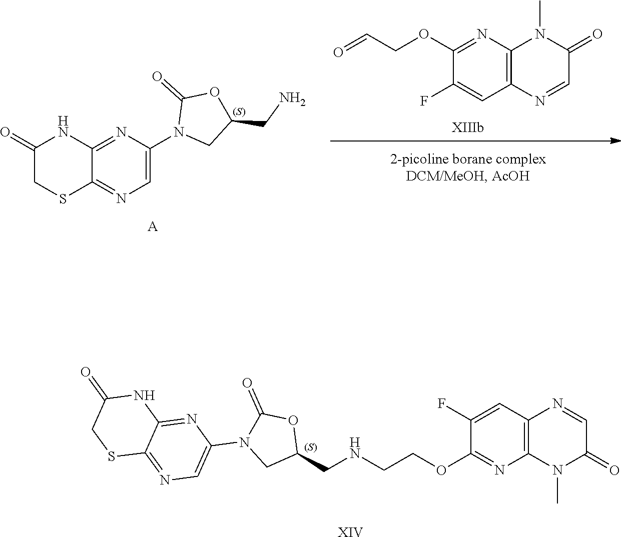

To a stirred mixture of XIIIb (0.45 g, 1.89 mmol) and compound A (WO2019186590, 0.65 g, 2.3 mmol) in a dry MeOH (40 mL)/DCM (40 mL) were added AcOH (0.5 mL) and 2-picoline borane complex (0.142 g, 1.32 mmol) at room temperature under nitrogen atmosphere. The resulting mixture was continued to stir at room temperature for 1 hour. After completion of the reaction mixture, reaction mixture was quenched with water (10 mL) and concentrated in vacuo. The crude product was purified by column chromatography by eluting with 5% methanol in dichloromethane to get compound XIV as a pale-orange solid. Yield: (0.13 g, 13.68%).

¹H NMR (300 MHz, DMSO-d₆): δ 11.20 (brs, 1H), 8.75 (s, 1H), 8.19-8.15 (m, 2H), 4.82-4.80 (m, 1H), 4.61-4.55 (m, 2H), 4.10-4.04 (m, 1H), 3.84-3.77 (m, 3H), 3.60 (s, 4H), 3.07-3.04 (t, J=7.20 Hz, 2H), 2.96-2.91 (m, 2H). LC_MS: Calc. for C₂₀H₁₉FN₈O₅S: 502.48; Obs.: 503.1 [M⁺+H].

### Synthesis of Comparative Compounds

### Compound 1: (S)-5-(((2-((4-Methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one

(Compound 1 was synthesized as per the reported procedure for compound 13 in patent WO2019186590 A1)

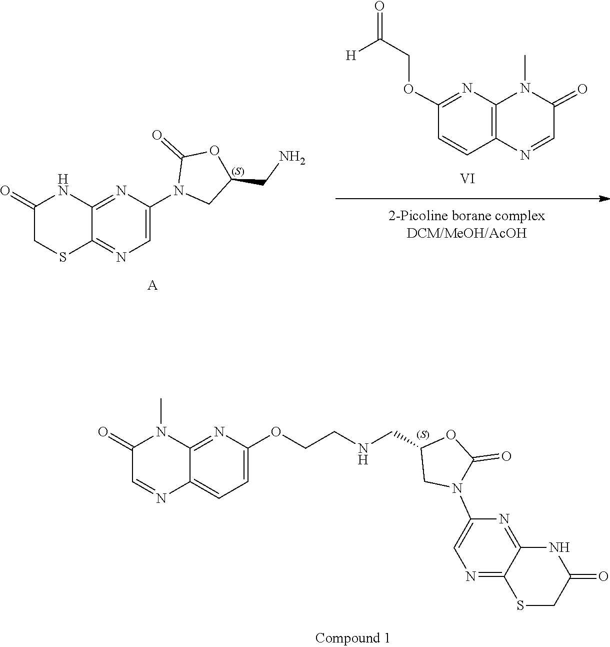

To a mixture of compound VI (0.2 g, 0.916 mmol) and compound A (WO2019186590, 0.257 g, 0.916 mmol) in a mixture of dry methanol (20 mL)/dichloromethane (20 mL) was added AcOH (0.40 mL) under nitrogen atmosphere at room temperature and allowed to stir for 16 hours. To this was added 2-picoline borane complex (0.058 g, 0.549 mmol) and stirred for another 15 minutes at room temperature. The reaction mixture was quenched with 1% HCOOH in water and concentrated under reduced pressure to get crude product. The crude product was purified by column chromatography using silica gel (230-400 mesh) eluting with 6% methanol in dichloromethane to afford the title compound as formate salt (Compound 1, pale yellow solid (0.100 g, 22.43%).

¹H NMR (400 MHz, DMSO-D₆): δ 11.68 (brs, 1H), 8.75 (s, 1H), 8.19 (brs, 1H), 8.09-8.07 (m, 2H), 6.79 (d, J=8.8 Hz, 1H), 4.83-4.79 (m, 1H), 4.48-4.44 (m, 2H), 4.10-4.05 (m, 1H), 3.84-3.80 (m, 1H), 3.75 (s, 3H), 3.59 (s, 3H), 3.01-2.94 (m, 4H). LC_MS: Calc. for C₂₀H₂₀N₈O₅S 484.49; Obs. 482.8 [M⁺−H]. HPLC: 98.55%; 2.18 min; HPLC Column: X-Bridge C18 (50*4.6) mm 3.5 μm, Mobile Phase A: 0.1% TFA in water, Mobile Phase B: Acetonitrile.

### Compound 2: (S)-5-(((2-((5-Methyl-6-oxo-5,6-dihydropyrido[2,3-b]pyrazin-3-yl)oxy)ethyl)amino)methyl)-3-(3-oxo-3,4-dihydro-2H-pyrazino[2,3-b][1,4]thiazin-6-yl)oxazolidin-2-one

(Compound 2 was synthesized as per the reported procedure for compound 14 in patent WO2019186590 A1)

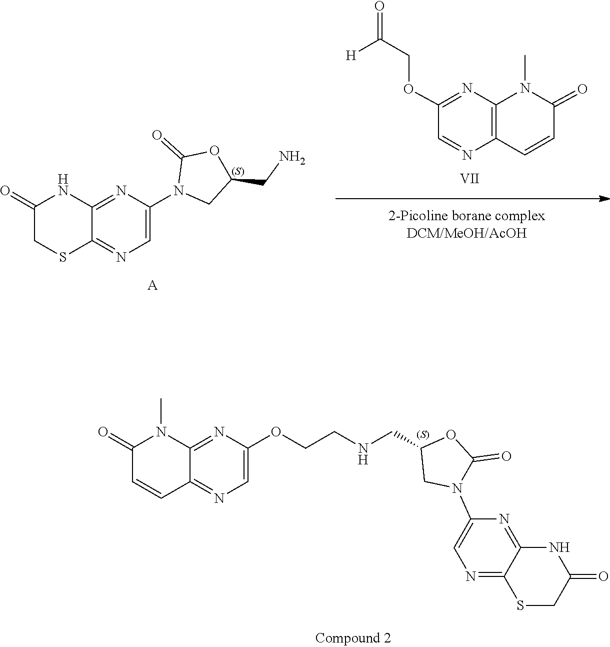

To a mixture of VII (0.17 g, 0.77 mmol) and A (0.24 g, 0.853 mmol) in a dry MeOH (20 mL)/DCM (20 mL) were added AcOH (0.2 mL) and 2-picoline borane complex (0.058 g, 0.54 mmol) at room temperature under nitrogen atmosphere. The resulting mixture was continued to stir at room temperature for 1 hour. After completion of the reaction mixture, reaction mixture was quenched with 1% HCOOH in water and concentrated in vacuo to get the crude. The crude was purified by column chromatography by eluting with 7% methanol in dichloromethane. The pure product obtained was further triturated with diethyl ether to afford pure product as formate salt (Compound 2, pale yellow solid, 25 mg, and 8.68%).

¹H NMR (400 MHz, DMSO-d₆): δ 11.23 (brs, 1H), 8.77 (s, 1H), 8.18-8.15 (m, 2H), 7.93 (d, J=12.00 Hz, 1H), 6.72 (d, J=12.00, Hz, 1H), 4.83 (s, 1H), 4.53-4.52 (m, 2H), 4.11-4.07 (m, 1H), 3.86-3.82 (m, 3H), 3.62 (s, 3H), 3.06-2.98 (m, 4H). LC_MS Calc. for C₂₀H₂₀N₈O₅S is 484.49; Obs. 484.9 [M⁺+H]; HPLC Purity=98.00%, X-Bridge C8 (50×4.6) mm, mm, 5 μm, Mobile Phase A: 0.1% TFA in water, Mobile Phase B: Acetonitrile.

### Synthesis of Compounds of the Present Disclosure

### Compound 3 (R isomer of compound 1): (R)-5-(((2-((4-methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one

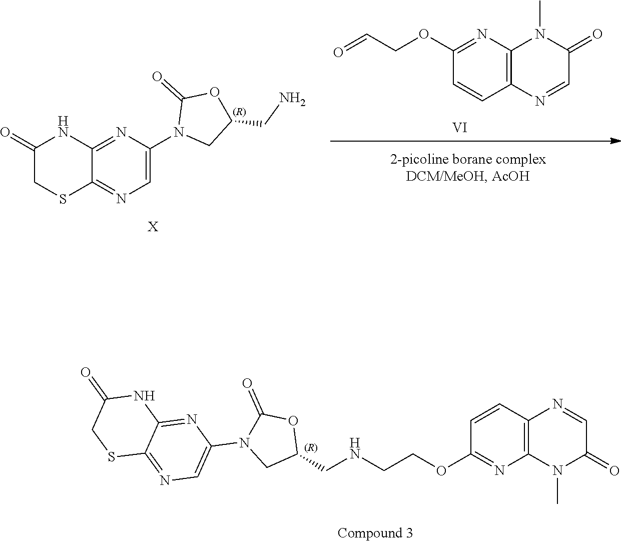

To a mixture of VI (5 g, 22.83 mmol) and X (7.05 g, 25.11 mmol) in a dry MeOH (100 mL)/DCM (100 mL) were added AcOH (5 mL) and 2-picoline borane complex (1.71 g, 15.98 mmol) at room temperature under nitrogen atmosphere. The resulting mixture was continued to stir at room temperature for 1 hour. After completion of the reaction mixture, reaction mixture was quenched with water (10 mL) and concentrated in vacuo. The crude product was purified three times by column chromatography by eluting with 5% methanol in dichloromethane. The product obtained was further triturated with diethyl ether to get Compound 3 as a pale-orange solid. Yield: (3.8 g, 37%)

¹H NMR (400 MHz, DMSO-d₆): δ 11.20 (brs, 1H), 8.76 (s, 1H), 8.10 (s, 1H), 8.08 (d, J=6 Hz, 1H), 6.79 (d, J=8.7 Hz, 1H), 5.76 (s, 1H), 4.84-4.77 (m, 1H), 4.49-4.42 (m, 2H), 4.08 (t, J=9 Hz, 1H), 3.85-3.80 (m, 1H), 3.76 (s, 2H), 3.59 (s, 3H), 3.02-2.89 (m, 4H). LC_MS: Calc. for C₂₀H₂₀N₈O₅S: 484.49; Obs.: 485.2 [M⁺+H]. HPLC Purity=98.11%, X-Bridge C8 (50×4.6) mm, 5 m, Mobile Phase A: 0.1% TFA in water, Mobile Phase B: Acetonitrile.

### Compound 4 (R isomer of compound 2): (R)-5-(((2-((5-methyl-6-oxo-5,6-dihydropyrido[2,3-b]pyrazin-3-yl) oxy) ethyl) amino) methyl)-3-(3-oxo-3,4-dihydro-2H-pyrazino[2,3-b][1,4]thiazin-6-yl)oxazolidin-2-one

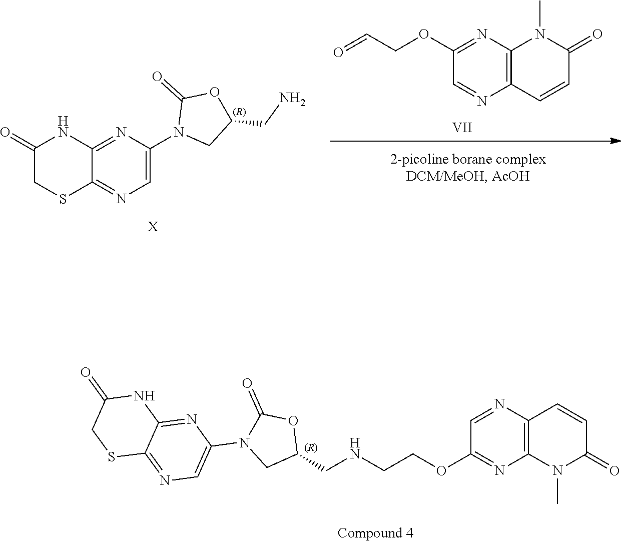

To a mixture of VII (4.5 g, 20.53 mmol) and X (8.66 g, 30.79 mmol) in a dry MeOH (225 mL)/CH2Cl2 (225 mL) at room temperature under nitrogen atmosphere was added acetic acid (9 mL, 2 vol) and stirred for 1 h. Then 2-picoline borane complex (1.53 g, 14.37 mmol) was added. The resulting mixture was continued to stir at room temperature for 1 hour. After completion of the reaction, reaction mixture was quenched with 1% HCOOH in water and concentrated in vacuo. The obtained crude product was purified by column chromatography by eluting with 7% methanol in dichloromethane. The product obtained was further triturated with diethyl ether to afford pure product as formate salt (Compound 4, pale-yellow solid). Yield: (4 g, 40.24%).

¹H NMR (400 MHz, DMSO-d₆): δ 11.19 (s, 1H), 8.76 (s, 1H), 8.17 (s, 1H), 7.90 (d, J=9.60 Hz, 1H), 6.71 (d, J=9.60 Hz, 1H), 5.76 (s, 1H), 4.83-4.80 (m, 1H), 4.51 (dd, J=9.20 Hz, J=5.2 Hz, 2H), 4.09 (dd, J=10.00 Hz, J=8.80 Hz, 1H), 3.84 (dd, J=10.00 Hz, J=6.4 Hz, 1H), 3.77 (s, 2H), 3.60 (s, 3H), 3.04 (t, J=5.6 Hz, 2H), 2.96 (t, J=4.8 Hz, 2H). LC_MS Calc. for C₂₀H₂₀N₈O₅S is 484.49; Obs. 484.9 [M⁺+H]. HPLC Purity=95.22%, X-Bridge C8 (50×4.6) mm, mm, 5 μm, Mobile Phase A: 0.1% TFA in water, Mobile Phase B: Acetonitrile.

### Compound 5: (R)-5-(((2-((4-methyl-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl)oxazolidin-2-one

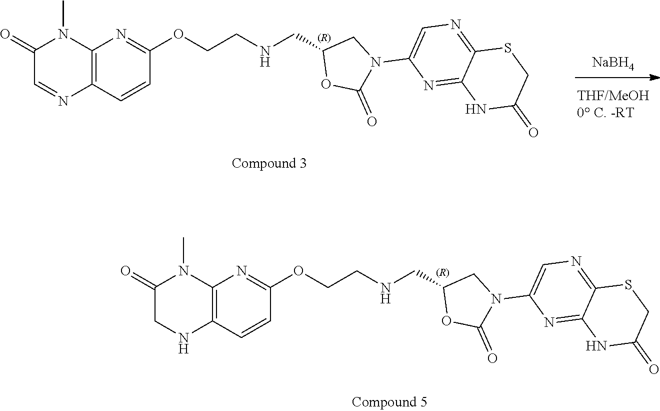

To a stirred solution of compound 3 (10 g, 0.0206 mol) in a mixture of THF: MeOH (1:1) (2.0 L) at 0° C. under nitrogen atmosphere was added NaBH₄ (1.73 g, 0.0454 mol). The reaction mixture was then stirred at room temperature of 2 hours. After completion of the reaction by TLC, the reaction mixture was quenched with water (50 mL) and concentrated in vacuo. The crude product was purified by column chromatography by eluting with 6% methanol in dichloromethane to get compound 5 as a pale-orange solid. Yield: (5 g, 50%)

¹H NMR (400 MHz, DMSO-d₆): δ 11.24 (brs, 1H), 8.80 (s, 1H), 7.06 (d, J=8.4 Hz, 1H), 6.31 (d, J=8.4 Hz, 1H), 5.76 (s, 1H), 4.84-4.78 (m, 1H), 4.21-4.18 (m, 2H), 4.08 (t, J=9 Hz, 1H), 3.87-3.78 (m, 1H), 3.76 (s, 4H), 3.33 (s, 3H), 2.93-2.90 (m, 4H). LC_MS: Calc. for C₂₀H₂₂N₈O₅S: 486.51; Obs.: 485.1 [M⁺−H]. HPLC Purity=96.75%, X-Bridge C8 (50×4.6) mm, 3.5 m, Mobile Phase A: 0.1% TFA in water, Mobile Phase B: Acetonitrile.

### Compound 6: (S)-5-(((2-((4-methyl-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl)oxazolidin-2-one

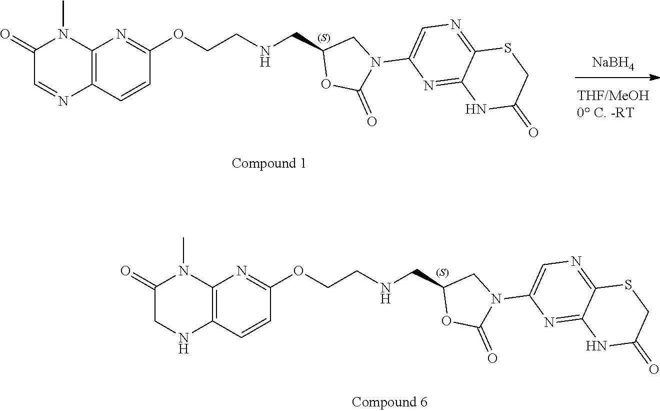

To a stirred solution of compound 1 (2 g, 4.1 mmol) in a mixture of THF:Methanol (1:1) (100 mL) at 0° C. under nitrogen atmosphere was added NaBH₄ (0.78 g, 21 mmol). The reaction mixture was then stirred at room temperature of 2 hours. After completion of the reaction by TLC, the reaction mixture was quenched with water (5 mL) and concentrated in vacuo. The crude product was purified by column chromatography by eluting with 8% methanol in dichloromethane to get compound 6 as a pale-orange solid. Yield: (1 g, 48%)

¹H-NMR (400 MHz, DMSO-d₆): δ 11.23 (br s, 1H), 8.81 (s, 1H), 7.07 (d, J=8.40 Hz, 1H), 6.32 (d, J=8.00 Hz, 1H), 4.37-4.20 (m, 1H), 4.19-4.09 (m, 4H), 3.87-3.61 (m, 1H), 3.28 (s, 4H), 2.90 (br s, 3H), 2.53-2.33 (m, 5H). LC_MS: Calc. for C₂₀H₂₂N₈O₅S: 486.51; Obs.: 487.0 [M⁺−H]. HPLC Purity=96.06%, X-Bridge C8 (50×4.6) mm, 3.5 μm, Mobile Phase A: 0.1% HCOOH in water, Mobile Phase B: Acetonitrile

### Compound 7: (R)-5-(((2-((7-fluoro-4-methyl-3-oxo-1,2,3,4-tetrahydropyrido[2,3-b]pyrazin-6-yl) oxy)ethyl)amino)methyl)-3-(3-oxo-3,4-dihydro-2H-pyrazino[2,3-b][1,4]thiazin-6-yl)oxazolidin-2-one

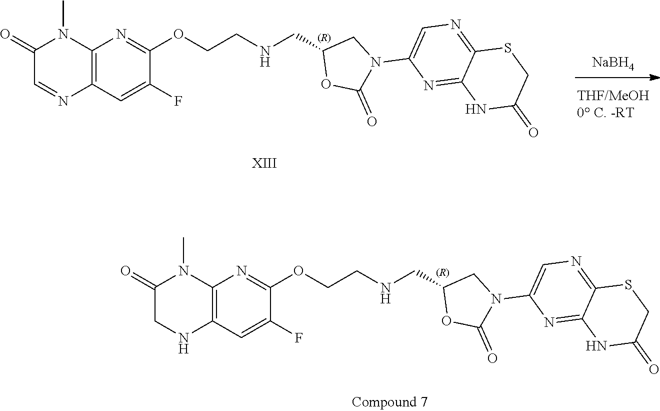

To a stirred solution of compound XIII (0.13 g, 0.2985 mol) in a mixture of THF:Methanol (1:1) (20 mL) at 0° C. under nitrogen atmosphere was added NaBH₄ (0.022 g, 0.5970 mol). The reaction mixture was then stirred at room temperature of 2 hours. After completion of the reaction by TLC, the reaction mixture was quenched with water (10 mL) and concentrated in vacuo. The crude product was purified by column chromatography by eluting with 6% methanol in dichloromethane to get Compound 7 as a pale-orange solid. Yield: (70 mg, 46.66%)

¹H NMR (300 MHz, DMSO-d₆): δ 11.21 (br s, 1H), 8.79 (s, 1H), 7.06 (d, J=10.80 Hz, 1H), 6.33 (br s, 1H), 4.85-4.79 (m, 1H), 4.30 (t, J=6.00 Hz, 2H), 4.14-4.08 (m, 1H), 3.87-3.77 (m, 5H), 3.21 (s, 3H), 2.52-2.51 (m, 4H), 2.50 (br s, 1H). LC_MS: Calc. for C₂₀H₂₁FN₈O₅S: 504.50; Obs.: 505.1 [M⁺+H]. HPLC Purity=97.18%, X-Bridge C8 (50×4.6) mm, 5 m, Mobile Phase A: 0.1% TFA in water, Mobile Phase B: Acetonitrile.

### Compound 8: (S)-5-(((2-((7-fluoro-4-methyl-3-oxo-1,2,3,4-tetrahydropyrido [2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one

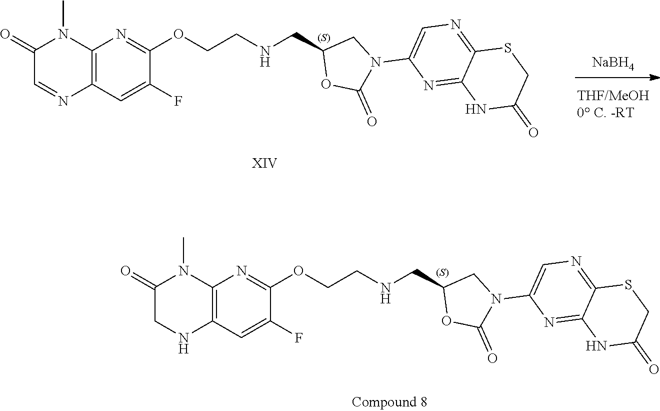

To a stirred solution of compound XIV (0.13 g, 0.2985 mol) in a mixture of THF:Methanol (1:1) (20 mL) at 0° C. under nitrogen atmosphere was added NaBH₄ (0.022 g, 0.5970 mol). The reaction mixture was then stirred at room temperature of 2 hours. After completion of the reaction by TLC, the reaction mixture was quenched with water (10 mL) and concentrated in vacuo. The crude product was purified by column chromatography by eluting with 6% methanol in dichloromethane to get compound 8 as a pale-orange solid. Yield: (75 mg, 57.69%).

¹H NMR (400 MHz, DMSO-d₆): δ 11.24 (brs, 1H), 8.81 (s, 1H), 7.05 (d, J=10.8 Hz, 1H), 5.94 (s, 1H), 4.84-4.79 (m, 1H), 4.31-4.28 (m, 2H), 4.09 (t, J=8.8 Hz, 1H), 3.86-3.81 (m, 3H), 3.76 (s, 2H), 3.26 (s, 3H), 2.96-2.93 (m, 4H). LC_MS: Calc. for C₂₀H₂₁FN₈O₅S: 504.50; Obs.: 505.1 [M⁺−H]. HPLC Purity=97.85%, X-Bridge C8 (50×4.6) mm, 3.5 m, Mobile Phase A: 0.1% TFA in water, Mobile Phase B: Acetonitrile.

### Compound 9: (R)-5-(((2-((4-(methyl-d3)-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl) oxazolidin-2-one

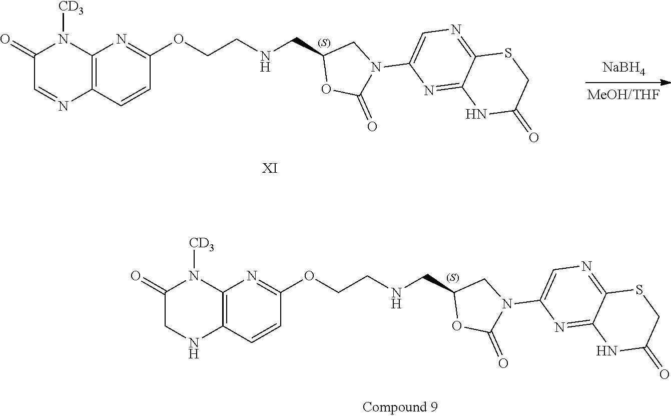

To a stirred solution of XI (0.62 g, 1.3 mmol) in a dry MeOH (20 mL)/THF (20 mL), cooled to 0° C. sodium borohydride (96 mg, 2.5 mmol) was added in portion wise under nitrogen atmosphere. The resulting mixture was continued to stir at room temperature for 2 hours. After completion of the reaction mixture, reaction mixture was neutralized with 1.5N HCl and concentrated to get crude as a yellow solid. The crude product was purified by column chromatography using silica gel (230-400 mesh) eluting with 7% methanol in dichloromethane to afford titled compound 9 as a yellow solid. Yield: (0.21 g, 33.8%).

¹H-NMR (400 MHz, DMSO-d₆): δ 11.23 (br s, 1H), 8.80 (s, 1H), 8.10 (d, J=5.60 Hz, 1H), 7.07 (d, J=8.40 Hz, 1H), 6.32 (s, 1H), 5.77 (br s, 1H), 4.83-4.81 (m, 1H), 4.39-4.36 (m, 1H), 4.22-4.19 (m, 2H), 4.14-4.09 (m, 2H), 3.87-3.83 (m, 1H), 3.78-3.77 (m, 4H), 2.96-2.91 (m, 2H). LC_MS: Calc. for C₂₀H₁₉D₃N₈O₅S: 489.53; Obs.: 490.1 [M⁺+H]. HPLC Purity=94.963%, X-Bridge C8 (50×4.6) mm, 3.5 m, Mobile Phase A: 0.1% HCOOH in water, Mobile Phase B: Acetonitrile.

### Compound 10 Synthesis of (S)-5-(((2-((4-Methyl-d₃)-3-oxo-1,2,3,4-tetrahydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one

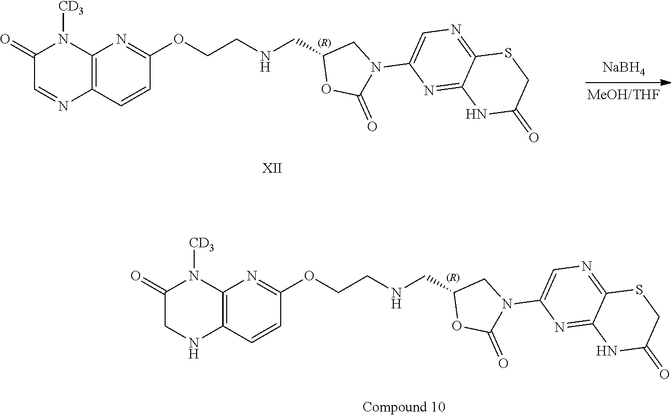

To a stirred solution of XII (0.2 g, 0.4 mmol) in a dry MeOH (10 mL)/THF (10 mL), cooled to 0° C. sodium borohydride (30 mg, 0.8 mmol) was added in portion wise under nitrogen atmosphere. The resulting mixture was continued to stir at room temperature for 2 hours. After completion of the reaction mixture, reaction mixture was neutralized with 1.5N HCl and concentrated to get crude as a yellow solid. The crude product was purified by column chromatography using silica gel (230-400 mesh) eluting with 7% methanol in dichloromethane to afford titled product compound 10 as a yellow solid. Yield: (90 mg, 45%).

¹H-NMR (400 MHz, DMSO-d₆): δ 11.21 (br s, 1H), 8.80 (s, 1H), 7.07 (d, J=8.40 Hz, 1H), 6.31 (d, J=8.00 Hz, 1H), 5.75 (s, 1H), 4.84-4.80 (m, 1H), 4.19 (t, J=19.60 Hz, 2H), 4.11-4.09 (m, 2H), 3.88-3.84 (m, 1H), 3.78-3.77 (m, 4H), 2.93 (br s, 4H), LC_MS: Calc. for C₂₀H₁₉D₃N₈O₅S: 489.53; Obs.: 490.1 [M⁺+H]. HPLC Purity=96.093%, X-Bridge C8 (50×4.6) mm, 3.5 m, Mobile Phase A: 0.1% HCOOH in water, Mobile Phase B: Acetonitrile.

### Example 4

### Biological Activity (Antibacterial Activity):

The compounds of Formula Ia and Formula Ib are of interest due to their potent antibacterial effects. The ability of the compounds disclosed herein to achieve an antibacterial effect may be evaluated with regard to their ability to inhibit the growth of bacterial species like Escherichia coli ATCC 25922, Staphylococcus aureus ATCC 29213, Klebsiella pneumoniae ATCC 13883, Acinetobacter baumannii ATCC 19606, Pseudomonas aeruginosa ATCC 27853 and Enterococcus faecalis ATCC 29212 using an assay based on the following Minimum Inhibitory Concentration (MIC) protocol:

The test bacteria were grown in Luria Bertani Broth (HIMEDIA M1245), 25 g of the powder was dissolved in 1000 ml distilled water and sterilized by autoclaving at 15 lbs pressure (121° C.) for 20 minutes. The medium sterility was checked by incubating at 37° C. for a period of 48 h. Bacterial cultures that were stored as glycerol stocks at −80° C. were subcultured on LB agar plates to obtain isolated colonies. A single colony of each strain was cultured in LB broth. The cultures were incubated at 37° C., 200 rpm till they reached an optical density (OD at 600 nm) of 0.8 to 1. This log phase culture was diluted in LB broth to a cell number of 5-8*10\hat{ }5 CFU/mL to be used as inoculum for MIC experiments. Test compounds were dissolved in dimethyl sulfoxide (DMSO) to a stock concentration of 4 mg/ml. A two-fold dilution series of this DMSO stock was prepared in a 96 well V bottom microtitre plate from rows A to H. A 3 μL volume of these dilutions are transferred to a 96-well flat bottom microtitre assay plate. Controls to monitor the effects of DMSO and media sterility were included. Each well was inoculated with 150 μL of the above diluted culture. The plates were incubated at 37° C. overnight in a humidified incubator. The following morning, the plates were read using a Spectrophotometer at 600 nM wavelength. Minimum Inhibitory Concentration (MIC) is defined as the lowest drug concentration well which showed no turbidity. The antibacterial activity (MIC) determined against representative gram-positive (S. aureus, E. faecalis) and gram-negative (E. coli, Aeruginosa, K. pneumoniae, and A. baumannii) pathogen are reported Table 1.

TABLE 1

| | Minimum Inhibitory Concentration (μg/mL) in LB Media | | | | | |
|---|---|---|---|---|---|---|
| | S. | | | | | A. |
| | aureus | E. faecalis | E. coli | Aeruginosa | K. | baumannii |
| | ATCC | ATCC | ATCC | ATCC | pneumoniae | ATCC |
| Compound | 29213 | 29212 | 25922 | 27853 | ATCC 13883 | 19606 |
| 1 | <0.03 | 0.1 | 0.03 | 0.5 | 0.03 | 0.03 |
| 2 | <0.03 | 0.03 | 0.03 | 0.5 | 0.03 | 0.03 |
| 3 | <0.015 | 0.03 | 0.03 | 0.5 | 0.03 | 0.03 |
| 4 | 0.015 | 0.06 | 0.06 | 0.5 | 0.06 | 0.03 |
| 5 | <0.015 | 0.125 | 0.03 | 1 | 0.06 | 0.06 |
| 6 | <0.015 | 0.06 | 0.06 | 0.5 | 0.06 | 0.06 |
| 7 | <0.015 | 0.06 | 0.03 | 1 | 0.06 | 0.03 |
| 8 | <0.015 | 0.125 | 0.03 | 1 | 0.06 | 0.06 |
| 9 | <0.015 | 0.03 | 0.06 | 1 | 0.06 | 0.03 |
| 10 | <0.015 | 0.125 | 0.06 | 1 | 0.06 | 0.06 |
| Ciprofloxacin | 0.4 | 0.4 | 0.012 | 0.1 | 0.05 | 0.4 |

### Example 5

Enzyme Inhibition Assay: Determination of IC₅₀ Against E. coli Gyrase Supercoiling and E. coli Topo IV Decatenation

The present disclosure also provided evidence for treating infection caused both by gram-positive and gram-negative bacteria through the inhibition of bacterial topoisomerases using E. coli DNA gyrase and E. coli Topo IV enzymes.

Procedure for E. coli DNA Gyrase Supercoiling Assay

E. coli gyrase supercoiling and its inhibition was assayed using a kit procured from Inpiralis (K0001) and the protocol (PMID: 2172086) was adapted with necessary modifications. The compounds to be tested were incubated for 10 minutes with 2.5 nM of E. coli DNA gyrase in a 30 μl reaction volume and 3.2% DMSO. The reactions were then started with the addition of 60 ng relaxed pBR322 plasmid DNA and continued for 45 min at 37° C. The reaction mixture contained 35 mM Tris-HCl (pH 7.5), 24 mM KCl, 1.8 mM spermidine, 4 mM MgCl₂, 2 mM DTT, 6.5% (w/v) glycerol, 0.1 mg/mL BSA, and 1 mM ATP. The reaction was then stopped by addition of 0.75 μL of Proteinase K (20 mg/mL) and 3 μL of 2% SDS and further incubated at 37° C. for 30 min. This was followed by the addition of 4 μL of STEB (40% (w/v) sucrose, 100 mM Tris-HCl (pH 8), 1 mM EDTA, 0.5 mg/ml Bromophenol Blue), and the supercoiled/relaxed forms of plasmid DNA were separated by agarose gel electrophoresis. The 1% agarose gels were run for 3 h at 4V/cm in 1×TAE (40 mM Tris, 20 mM Acetic acid, 1 mM EDTA). To visualize the DNA, the gels were stained for 10 min with 0.7 μg/mL ethidium bromide and excess dye was removed by several washes with water. IC₅₀ values were determined by quantifying the supercoiled and relaxed DNA in each of the reactions from a gel image by a densitometric method using the Quantity One Software (Bio-rad).

Procedure for E. coli Topoisomerase IV Decatenation Assay

E. coli topoisomerase IV decatenation activity and its inhibition was assayed using a kit procured from Inpiralis (D4002) and the kit protocol was adapted with necessary modifications similar to the gyrase supercoiling assays. The compounds 1, 2, 3 and 4 were incubated individually for 10 minutes with 5 nM of E. coli topoisomerase IV in a 30 μl reaction volume and 3.2% DMSO. The reactions were started with the addition of 60 ng of kDNA and continued for 40 min at 37° C. The final reaction mixture contained 40 mM Tris-HCl (pH 7.6), 100 mM potassium glutamate, 10 mM magnesium acetate, 10 mM DTT, 1 mM ATP, and 50 μg/ml albumin. The reactions were stopped by addition of 0.75 μL of Proteinase K (20 mg/mL) and 3 μL of 2% SDS and further incubated at 37° C. for 30 min. This was followed by the addition of 4 μL of STEB (40% (w/v) sucrose, 100 mM Tris-HCl pH8, 1 mM EDTA, 0.5 mg/ml Bromophenol Blue) and the kDNA/minicircles forms were separated by agarose gel electrophoresis. The 1% agarose gels were run for 3 h at 4V/cm in 1×TAE (40 mM Tris, 20 mM Acetic acid, 1 mM EDTA). To visualize the DNA, the gels were stained for 10 min with 0.7 μg/mL ethidium bromide and excess dye was removed by several washes with water. IC₅₀ values were determined by quantifying the Kinetoplast DNA band inside the gel well and decatenated minicircles that migrate into the gel in each of the reactions from a gel image by a densitometric method using the Quantity One Software (Bio-rad).

Representing examples belonging to Formula Ia and Formula Ib were evaluated against of E. coli DNA gyrase and Topo IV enzyme using gel based supercoiling assay for gyrase inhibition and decatenation assay for Topo IV inhibition. The results for bacterial Type II Topoisomerases (Gyrase and Topo IV), presented in the Table 2 indicated that compounds belonging to Formula Ia and Formula Ib exert their antibacterial activity through inhibition of bacterial type II topoisomerase activity and confirmed the dual mode of inhibition for observed antibacterial activity of the compounds.

TABLE 2

| | E. coli DNA | E. coli Topo |
|---|---|---|
| Compound | Gyrase IC₅₀ (μM) | IV IC₅₀ (μM) |
| 1 | 0.025 | 0.050 |
| 2 | 0.013 | 0.045 |
| 3 | 0.03 | 0.05 |
| 4 | 0.025 | 0.044 |
| 5 | 0.32 | 0.11 |
| 6 | 0.12 | 0.13 |
| 7 | 0.067 | ND |
| 8 | 0.095 | ND |
| 9 | 0.17 | 0.091 |
| 10 | 0.07 | 0.078 |
| Ciprofloxacin | 0.233 | 14.4 |

ND—not determined

### Example 6

### MIC₉₀ Determination

To test if the compounds of Formula Ia and Formula Ib were able to retain the antibacterial activity against multi-drug resistant clinical strains of bacteria, antibacterial susceptibility studies (MIC₉₀ determination) were carried out for the compounds 1 to 4 and 10 from the series using clinical strains of eleven gram-negative bacterial species (Acinetobacter baumannii, Citrobacter freundii species complex, Enterobacter cloacae species complex, Escherichia coli, Klebsiella aerogenes, Klebsiella oxytoca, Klebsiella pneumoniae, Morganella morganii, Proteus mirabilis, Providencia rettgeri, Providencia stuartii, Pseudomonas aeruginosa and Serratia marcescens) according to the standard CLSI guidelines and the results obtained are presented Table 3. The standard drugs ciprofloxacin and meropenem were taken as positive control in the study. As it can be observed from Table 3, both the compounds 3, 4, and 10 exhibited comparable MIC values for all the bacterial strains. Hence, it was clear that the compounds of Formula Ia and Formula Ib are useful in inhibiting the growth of bacteria.

TABLE 3
MIC₉₀ results

| | Number | | | | | | | |
|---|---|---|---|---|---|---|---|---|
| | of | MIC 90 (μg/ml) | | | | | | |
| | strains | Compound | Compound | Compound | Compound | Compound | | |
| Bacterial species | (N) | 1 | 2 | 3 | 4 | 10 | Ciprofloxacin | Meropenem |
| Acinetobacter | 38 | 0.5 | 2 | 0.5 | 1 | 1 | >128 | >8 |
| baumannii | | | | | | | | |
| Citrobacter freundii | 19 | 2 | 1 | 1 | 2 | 2 | 128 | 8 |
| Enterobacter | 26 | 4 | 8 | 8 | 8 | 8 | 64 | 16 |
| cloacae | | | | | | | | |
| Escherichia coli | 38 | 0.5 | 1 | 0.5 | 0.5 | 0.5 | 128 | 2 |
| Klebsiella | 17 | 2 | 2 | 4 | 4 | 2 | 32 | 16 |
| aerogenes, | | | | | | | | |
| K. oxytoca | | | | | | | | |
| Klebsiella | 86 | 4 | 4 | 4 | 8 | 8 | >128 | 16 |
| pneumoniae | | | | | | | | |
| Morganella | 11 | 1 | 1 | 1 | 2 | 1 | 128 | 0.12 |
| morganii | | | | | | | | |
| Proteus mirabilis | 12 | 4 | 4 | 4 | 4 | 4 | >128 | 1 |
| Providencia | 12 | 2 | 4 | 2 | 2 | 1 | 64 | 4 |
| rettgeri, P. stuartii | | | | | | | | |
| Pseudomonas | 34 | 2 | 4 | 4 | 4 | 4 | 32 | 16 |
| aeruginosa | | | | | | | | |
| Serratia marcescens | 15 | 8 | 16 | 4 | 16 | 16 | 128 | 16 |

### Example 7

### In-Vitro Micronucleus Assay

Genetic toxicology studies were performed on the compounds of the present disclosure to assess their ability to cause DNA damage. The in-vitro micronucleus assay measured the concentration (μM) of the compound at which micronuclei formation occurred. A micronuclei formation at lower concentration indicates more genetic damage or genotoxicity.

Micronucleus formation is a hallmark of genotoxicity, and the micronucleus assay is an important component of genotoxicity screening. Micronuclei are chromatin-containing bodies that represent fragments or even whole chromosomes that were not incorporated into a daughter cell nucleus at mitosis. The purpose of the assay is to detect agents that induce chromosome damage leading to the induction of micronuclei in interphase cells. The in vitro micronucleus assay was conducted in CHO-K1 cells according to the procedure described by Diaz, D et al [Mutat Res. 630(1-2):1-13. 2007]. The formation of the micronucleus is identified by image-based high content analysis from binucleated cells.

The results obtained from the in-vitro micronucleus assay are presented in Table 4. It can be clearly inferred from the generated data that both the compounds 1 and 2 exhibited genotoxicity at concentration of 32 and 8 μM respectively. On the other hand, compound 4 induced in-vitro micro nucleus formation at appreciably higher concentrations of 130 μM and compound 3, 5, 6 did not induce micronucleus formation even at the highest concentrations tested (>500 or 1000 μM). The compounds 3, 4, 5, 6 have the similar biological activity as that of compound 1 and 2 but have surprisingly different pattern as far as genotoxicity is concerned. Specifically compounds of the present disclosure 3, 5, 6, 7, 8, 9 and 10 were completely devoid of any genotoxicity risk. Thus, the chirality of at C-4 carbon of oxazolidinone ring (in case of compounds 3 and 4) or saturation of double bond of the pyrazinone part of quinazolinone LHS ring (in case of 5 and 6) seems to be playing the major role in terms of mitigating the genotoxicity risk. In addition, a minor role of the substituents pattern in the LHS ring (position of nitrogen) is also observed for reducing the genotoxicity risk.

TABLE 4
In-vitro micronucleus assay results

| Compound | In vitro micronucleus forming | % |
|---|---|---|
| 1 (Compound 13, | 32 | 1.58 |
| WO2019186590) | | |
| 2 (Compound 14, | 8 | 1.67 |
| WO2019186590) | | |
| 3 | >500 | 0.81 |
| 4 | >130 | 0.76 |
| 5 | >1000 | 0.5 |
| 6 | >1000 | 0.4 |
| 7 | >1000 | 0.41 |
| 8 | >1000 | 0.5 |
| 9 | >1000 | 0.51 |
| 10 | >1000 | 0.45 |
| Control -S9 | — | 0.38 |
| Mitomycin C (positive control) | 0.300 | 1.98 |

### Example 8

hERG (Human Ether a go-go Related Gene) Inhibition Assay

To understand if the compounds of Formula Ia and Ib have any safety risk by inhibiting cardiac ion channel, particularly the potassium channel (Ikr, hERG), compounds were tested using electrophysiological assays to evaluate its potential activity on hERG ion channel. The representative compounds of Formula Ia and Ib were tested for inhibition of the human ether a go-go related gene (hERG) K+ channel using Qpatch HTX automated electrophysiology. 6-Point concentration-response curves were generated using three-fold serial dilutions from a maximum final test concentration of 300 μM and the results are presented in Table 5.

Compounds were solubilised to 100 mM in DMSO before dilution in HBPS to 100 μM. 6-Point concentration-response curves were generated using 3.16-fold serial dilutions from the top test concentration. Electrophysiological recordings were made from a Chinese Hamster Ovary cell line stably expressing the full-length hERG potassium channel. Single cell ionic currents were measured in whole-cell patch clamp configuration at room temperature (21-23° C.) using the Qpatch HTX platform (Sophion). Intracellular solution contained (mM): 120 KF, 20 KCl, 10 EGTA, 10 HEPES and was buffered to pH 7.3. The extracellular solution (HEPES-buffered physiological saline, HBPS) contained (mM): 145 NaCl, 4 KCl, 2 CaCl₂, 1 MgCl₂, 10 HEPES, 10 glucose, buffered to pH 7.4. Cells were clamped at a holding potential of −80 mV. Cells were stepped to +20 mV for 2 s then −40 mV for 3 s before returning to the holding potential. This sweep was repeated 10 times at 10 s intervals. hERG currents were measured from the tail step and referenced to the holding current. Compounds were then incubated for 2 minutes prior to a second measurement of ion channel current using an identical pulse train.

TABLE 5
hERG IC₅₀ values

| Example | hERG IC₅₀ (μM) |
|---|---|
| 1 | 58 |
| 2 | >150 |
| 4 | >300 |
| 5 | >300 |
| 6 | >300 |
| 7 | >300 |
| 8 | >300 |
| 9 | 141 |
| 10 | 157 |
| Cisapride | 0.14 |
| (positive | |
| control) | |

It can be clearly inferred from table 5 that compounds of the present disclosure demonstrate high degree of selectivity against hERG channel (cardiac potassium ion channel) over compound 1 and may be devoid of cardio toxicity in animal and human. Cisapride is a known hERG ion channel inhibitor and used as a positive control for the hERG inhibition assay.

### Advantages of the Present Disclosure

The above-mentioned implementation examples as described on this subject matter and its equivalent thereof have many advantages, including those which are described.

The compounds of the present disclosure exhibit excellent antimicrobial activity against a wide spectrum of gram-positive and gram-negative bacterial strains, with much potent MIC values. Hence, they can be administered at lower in achieving antibacterial effects. With highly reduced in-vitro micronucleus formation, the present compounds are also safer to use in terms of DNA damage and genotoxicity. The compounds of the present disclosure are effective antibiotics, especially the specific enantiomeric form of the compounds are highly efficient in inhibiting bacterial growth. The specific isomeric forms are found to exhibit little or no genotoxicity, hence proving the significance of the structural aspects of the compounds in its isomeric forms. Thus, the compounds of the present disclosure are potential candidates for the manufacture of medicaments used for treating infectious diseases caused by various disease-causing microorganisms.

Although the subject matter has been described in considerable detail with reference to certain embodiments thereof, other embodiments are possible. As such, the spirit and scope of the disclosure should not be limited to the description of the embodiments contained herein.

I/We claim:

1. A compound selected from Formula Ia or Formula Ib 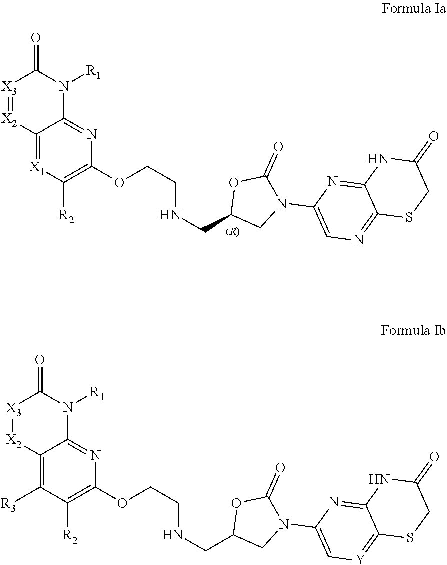
its stereoisomers, pharmaceutically acceptable salts, complexes, hydrates, solvates, tautomers, polymorphs, or pharmaceutically active derivatives thereof,
wherein
R₁ is selected from C₁₋₆ alkyl, C₂₋₆ alkenyl, C₃₋₆ cycloalkyl, CD₃, C₁₋₆ alkoxy, C₁₋₆ haloalkyl, or C₁₋₆ haloalkoxy;
R₂ is selected from hydrogen, C₁₋₆ alkyl, halogen, hydroxy, or amino;
R₃ is selected from hydrogen, halogen, hydroxyl, amino, cyano, C₁₋₆ alkoxy, C₁₋₆ haloalkyl, C₁₋₆ haloalkoxy, C₁₋₆ alkyl, NH—R₄, or —CH₂CH₂OH;
R₄ is selected from C₁₋₆ alkyl, C₃₋₆ cycloalkyl, C₁₋₆ alkoxy, C₁₋₆ haloalkyl, C₁₋₆ haloalkoxy —CH₂CH₂OH, or —CH₂CH₂NH₂;
X₁ is N or CR₃;
X₂ is CR₅, O, N, or NR₆ when X₃ is CH or CH₂;
R₅ is selected from hydrogen, cyano, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl;
R₆ is selected from hydrogen, C₁₋₆ alkyl, C₁₋₆ alkylamino, C₁₋₆ alkoxy, or C₁₋₆ haloalkoxy, wherein C₁₋₆ alkyl, and C₁₋₆ alkylamino are optionally substituted with one or more groups selected from hydroxyl, amino, or C₁₋₆ alkyl;
X₃ is N or NR₇ when X₂ is CH₂ or CR₅;
R₇ is selected from hydrogen, or C₁₋₆ alkyl;
Y is N or CR₈; and
R₈ is selected from hydrogen, halogen, cyano, C₁₋₆ alkyl, C₁₋₆ alkoxy, or C₁₋₆ haloalkoxy.

2. The compound as claimed in claim 1, its stereoisomers, pharmaceutically acceptable salts, complexes, hydrates, solvates, tautomers, polymorphs, or pharmaceutically active derivatives thereof, wherein R₁ is selected from C₁₋₆ alkyl, or CD₃; R₂ is hydrogen or halogen; R₃ is selected from hydrogen or C₁₋₆ alkyl; X₁ is N or CR₃; X₂ is CR₅, N, or NR₆ then X₃ is CH or CH₂; R₅ is selected from hydrogen, or C₁₋₆ alkyl; R₆ is selected from hydrogen, or C₁₋₆ alkyl; Y is N or CR₈; and R₈ is selected from hydrogen, halogen, cyano, or C₁₋₆ alkyl.

3. The compound as claimed in claim 1, its pharmaceutically acceptable salts, complexes, hydrates, solvates, tautomers, polymorphs, or pharmaceutically active derivatives thereof, wherein the compound is selected from:
i. (R)-5-(((2-((4-methyl-3-oxo-3,4-dihydropyrido[2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one; and
ii. (R)-5-(((2-((5-methyl-6-oxo-5,6-dihydropyrido[2,3-b]pyrazin-3-yl) oxy) ethyl) amino) methyl)-3-(3-oxo-3,4-dihydro-2H-pyrazino[2,3-b][1,4]thiazin-6-yl)oxazolidin-2-one.

4. The compound as claimed in claim 1, its pharmaceutically acceptable salts, complexes, hydrates, solvates, tautomers, polymorphs, or pharmaceutically active derivatives thereof, wherein the compound is selected from:
i. (R)-5-(((2-((4-methyl-3-oxo-1,2,3,4-tetrahydropyrido[2,3-b]pyrazin-6-yl)oxy) ethyl)amino)methyl)-3-(3-oxo-3,4-dihydro-2H-pyrazino [2,3-b][1,4]thiazin-6-yl)oxazolidin-2-one;
ii. (S)-5-(((2-((4-methyl-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl)oxazolidin-2-one;
iii. (R)-5-(((2-((7-fluoro-4-methyl-3-oxo-1,2,3,4-tetrahydropyrido[2,3-b]pyrazin-6-yl) oxy)ethyl)amino)methyl)-3-(3-oxo-3,4-dihydro-2H-pyrazino[2,3-b][1,4]thiazin-6-yl)oxazolidin-2-one;
iv. (S)-5-(((2-((7-fluoro-4-methyl-3-oxo-1,2,3,4-tetrahydropyrido [2,3-b]pyrazin-6-yl)oxy)ethyl)amino)methyl)-3-(3-oxo-3,4-dihydro-2H-pyrazino[2,3-b][1,4]thiazin-6-yl)oxazolidin-2-one;
v. (R)-5-(((2-((4-(methyl-d3)-3-oxo-1,2,3,4-tetrahydropyrido[2,3-b]pyrazin-6-yl)oxy) ethyl)amino)methyl)-3-(3-oxo-3,4-dihydro-2H-pyrazino[2,3-b][1,4]thiazin-6-yl) oxazolidin-2-one; and
vi. (S)-5-(((2-((4-(methyl-d3)-3-oxo-1,2,3,4-tetrahydropyrido[2,3-b]pyrazin-6-yl) oxy) ethyl)amino)methyl)-3-(3-oxo-3,4-dihydro-2H-pyrazino[2,3-b][1,4]thiazin-6-yl) oxazolidin-2-one.

5. A process of preparation of compound of Formula Ia as claimed in claim 1, its pharmaceutically acceptable salts, complexes, hydrates, solvates, tautomers, polymorphs, or pharmaceutically active derivative thereof, said process comprising reacting Formula (X) with Formula (VI) in presence of at least one reducing agent to obtain the compound of Formula Ia.

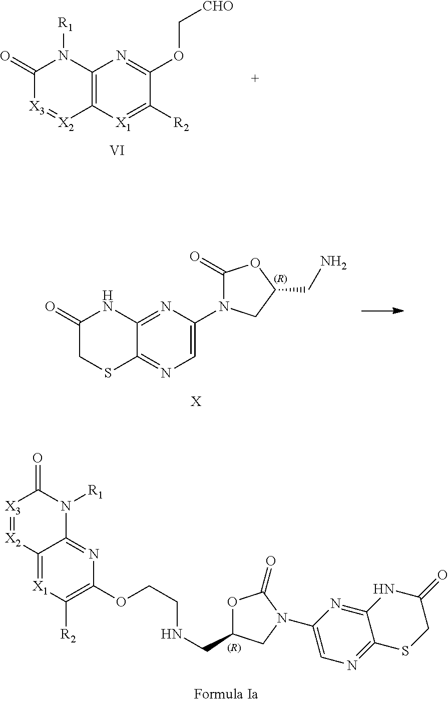

6. A process of preparation of compound of Formula Ib as claimed in claim 1, its stereoisomers, pharmaceutically acceptable salts, complexes, hydrates, solvates, tautomers, polymorphs, or pharmaceutically active derivative thereof, said process comprising reacting Formula (XI) with Formula (VII) in presence of at least one reducing agent to obtain the compound of Formula Ib.

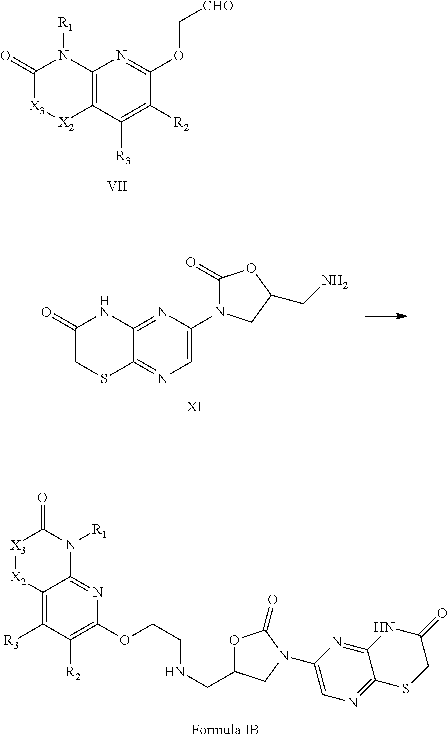

7. The process as claimed in claims 5 and 6, wherein the at least one reducing agent is selected from the group consisting of 2-picoline borane complex, sodium borohydride, sodium cyano borohydride, sodium triacetoxy borohydride, and combinations thereof.

8. The compound as claimed in any one of the claims 1 to 4, its stereoisomers, pharmaceutically acceptable salts, complexes, hydrates, solvates, tautomers, polymorphs, or pharmaceutically active derivatives thereof, for use as a medicament.

9. The compound as claimed in any one of the claims 1 to 4, its stereoisomers, pharmaceutically acceptable salts, complexes, hydrates, solvates, tautomers, polymorphs, or pharmaceutically active derivative thereof, for use in killing or inhibiting the growth of a microorganism selected from bacteria, virus, fungi, and protozoa.

10. The compound as claimed in any one of the claims 1 to 4, its stereoisomers, pharmaceutically acceptable salts, complexes, hydrates, solvates, tautomers, polymorphs, or pharmaceutically active derivative thereof, for use in treatment of a bacterial infection caused by a Gram-positive bacterium or a Gram-negative bacterium.

11. The compound as claimed in any one of the claims 1 to 4, its stereoisomers, pharmaceutically acceptable salts, complexes, hydrates, solvates, tautomers, polymorphs, or pharmaceutically active derivative thereof, for use in treating a disease or condition in a patient wherein said disease or condition is caused by a microorganism selected from the group consisting of Gram-positive, and Gram-negative pathogens.

12. A pharmaceutical composition comprising a compound as claimed in any one of the claims 1 to 4, its stereoisomers, pharmaceutically acceptable salts, complexes, hydrates, solvates, tautomers, polymorphs, or pharmaceutically active derivative thereof together with a pharmaceutically acceptable carrier, optionally in combination with at least one antibiotic.

13. A pharmaceutical composition comprising a compound selected from Formula Ta or Formula Tb as claimed in claim 1, its stereoisomers, pharmaceutically acceptable salts, complexes, hydrates, solvates, tautomers, polymorphs, or pharmaceutically active derivative thereof, wherein the compound of Formula Ta or Formula Ib has an enantiomeric excess in the range of 95%-99.9%

14. Use of compound as claimed in any one of the claims 1 to 4, its stereoisomers, pharmaceutically acceptable salts, complexes, hydrates, solvates, tautomers, polymorphs, or pharmaceutically active derivative thereof, in killing or inhibiting the growth of a microorganism selected from the group consisting of bacteria, virus, fungi, and protozoa.

15. A method for treatment of bacterial infection in a subject comprising: administering to the subject an effective amount of the compound as claimed in any one of the claims 1 to 4.

16. The method as claimed in claim 15, wherein the bacterial infection is caused by a Gram-positive or a Gram-negative pathogen.

17. The method as claimed in claim 16, wherein the bacterial infection is caused by E. coli, Pseudomonas aeruginosa, Klebsiella pneumoniae, Acinetobacter baumannii, Enterobacter cloacae, Staphylococcus aureus, Enterococcus faecalis Enterococcus faecium, Legionella pneumophila. Mycoplasma pneumonia, Acinetobacter haemolyticus Acinetobacter junii, Acinetobacter lwoffi, Burkholderia cepacia, Chlamydophila pneumoniae, Clostridium difficili, Enterobacter aerogenes, Enterobacter cloacae. Moraxella catarrhalis, Neisseria gonorrhoeae, Neisseria meningitides, Proteus mirabilis, Proteus houseri, Citrobacter freundii, Citrobacter kosari, Citrobacter barakii, Seratia marcescens, Klebsiella oxytoca, Morganella morganii, Helicobacter pyroli, or Mycobacterium tuberculosis.
